SEQUENCE LISTING (23 sequences)

SEQ ID NO: 1 - protein, 453 aa, synthetic construct
  REGION 1..453: note = 18D1-HC(N297A)
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 704 aa, synthetic construct
  REGION 1..704: note = 18D1-HC(N297A)-scFv:18D1
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEEVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLG

SEQ ID NO: 3 - protein, 223 aa, synthetic construct
  REGION 1..223: note = 18D1-LC
QSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLGGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 474 aa, synthetic construct
  REGION 1..474: note = 18D1-LC-scFv:18D1
QSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLGGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEEVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLG

SEQ ID NO: 5 - protein, 361 aa, synthetic construct
  REGION 1..361: note = scFv:18D1-CL
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLGGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 583 aa, synthetic construct
  REGION 1..583: note = scFv:18D1-CH1,2,3(N297A)
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLGGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 612 aa, synthetic construct
  REGION 1..612: note = scFv:18D1-CL-scFv:18D1
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLGGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEEVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLG

SEQ ID NO: 8 - protein, 834 aa, synthetic construct
  REGION 1..834: note = scFv:18D1-CH1,2,3(N297A)-scFv:18D1
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLGGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEEVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLG

SEQ ID NO: 9 - protein, 453 aa, synthetic construct
  REGION 1..453: note = PDL192-HC(N297A)
EVQLVESGGGLVQPGGSLRLSCAASGFTFSSYWMSWVRQAPGKGLEWVAEIRLKSDNYATHYAESVKGRFTISRDDSKNSLYLQMNSLRAEDTAVYYCTGYYADAMDYWGQGTLVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 704 aa, synthetic construct
  REGION 1..704: note = PDL192-HC(N297A)-scFv:PDL192
EVQLVESGGGLVQPGGSLRLSCAASGFTFSSYWMSWVRQAPGKGLEWVAEIRLKSDNYATHYAESVKGRFTISRDDSKNSLYLQMNSLRAEDTAVYYCTGYYADAMDYWGQGTLVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFEVQLVESGGGLVQPGGSLRLSCAASGFTFSSYWMSWVRQAPGKGLEWVAEIRLKSDNYATHYAESVKGRFTISRDDSKNSLYLQMNSLRAEDTAVYYCTGYYADAMDYWGQGTLVTVSSRSSTKGPKLEEGEFSEAQLDIQMTQSPSSLSASVGDRVTITCRASQSVSTSSYSYMHWYQQKPGKAPKLLIKYASNLESGVPSRFSGSGSGTDFTLTISSLQPEDFATYYCQHSWEIPYTFGGGTKVEIK

SEQ ID NO: 11 - protein, 218 aa, synthetic construct
  REGION 1..218: note = PDL192-LC
DIQMTQSPSSLSASVGDRVTITCRASQSVSTSSYSYMHWYQQKPGKAPKLLIKYASNLESGVPSRFSGSGSGTDFTLTISSLQPEDFATYYCQHSWEIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 469 aa, synthetic construct
  REGION 1..469: note = PDL192-LC-scFv:PDL192
DIQMTQSPSSLSASVGDRVTITCRASQSVSTSSYSYMHWYQQKPGKAPKLLIKYASNLESGVPSRFSGSGSGTDFTLTISSLQPEDFATYYCQHSWEIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEFEVQLVESGGGLVQPGGSLRLSCAASGFTFSSYWMSWVRQAPGKGLEWVAEIRLKSDNYATHYAESVKGRFTISRDDSKNSLYLQMNSLRAEDTAVYYCTGYYADAMDYWGQGTLVTVSSRSSTKGPKLEEGEFSEAQLDIQMTQSPSSLSASVGDRVTITCRASQSVSTSSYSYMHWYQQKPGKAPKLLIKYASNLESGVPSRFSGSGSGTDFTLTISSLQPEDFATYYCQHSWEIPYTFGGGTKVEIK

SEQ ID NO: 13 - protein, 361 aa, synthetic construct
  REGION 1..361: note = scFv:PDL192-CL
EVQLVESGGGLVQPGGSLRLSCAASGFTFSSYWMSWVRQAPGKGLEWVAEIRLKSDNYATHYAESVKGRFTISRDDSKNSLYLQMNSLRAEDTAVYYCTGYYADAMDYWGQGTLVTVSSRSSTKGPKLEEGEFSEAQLDIQMTQSPSSLSASVGDRVTITCRASQSVSTSSYSYMHWYQQKPGKAPKLLIKYASNLESGVPSRFSGSGSGTDFTLTISSLQPEDFATYYCQHSWEIPYTFGGGTKVEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 583 aa, synthetic construct
  REGION 1..583: note = scFv:PDL192-CH1,2,3(N297A)
EVQLVESGGGLVQPGGSLRLSCAASGFTFSSYWMSWVRQAPGKGLEWVAEIRLKSDNYATHYAESVKGRFTISRDDSKNSLYLQMNSLRAEDTAVYYCTGYYADAMDYWGQGTLVTVSSRSSTKGPKLEEGEFSEAQLDIQMTQSPSSLSASVGDRVTITCRASQSVSTSSYSYMHWYQQKPGKAPKLLIKYASNLESGVPSRFSGSGSGTDFTLTISSLQPEDFATYYCQHSWEIPYTFGGGTKVEIKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 490 aa, synthetic construct
  REGION 1..490: note = 18D1-HC(N297A)-TNC
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 16 - protein, 453 aa, synthetic construct
  REGION 1..453: note = 18D1-HC(A+RGY)
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 17 - protein, 620 aa, synthetic construct
  REGION 1..620: note = scFv:18D1- CH1,2,3(N297A)-TNC
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLGGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 18 - protein, 529 aa, synthetic construct
  CONFLICT 1..529: note = scFv:18D1-TNC- Fc(N297A)
EVQLVESGGGLVQPGGSLRLSCAASGFTFSNYWMSWVRQAPGKGLEWVSGINPGGTSTYYADSVKGRFTISRDNAKNTLYLQMNSLKSEDTAVYYCAKHLGNWGEYNYWGQGTQVTVSSRSSTKGPKLEEGEFSEAQLQSALTQPPSVSGSPGKTVTISCAGTGGDVGYRNSVSWYQQLPGMAPKLLIYDVDKRASGITDRFSGSKSGDTASLTISGVQSEDEADYYCASQRSGIAAVFGGGTHLTVLGGSDYKDDDDKDIACGCAAAPDIKDLLSRLEELEGLVSSLREQGTGGGSGGRGEFKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 26 aa, synthetic construct
  REGION 1..26: note = Leader sequence
MNFGFSLIFLVLVLKGVQCEVKLVPR

SEQ ID NO: 20 - protein, 8 aa, synthetic construct
  REGION 1..8: note = flag tag
DYKDDDDK

SEQ ID NO: 21 - protein, 35 aa, synthetic construct
  REGION 1..35: note = Tenascin C (TNC) trimerization domain
DIACGCAAAPDIKDLLSRLEELEGLVSSLREQGTG

SEQ ID NO: 22 - protein, 19 aa, synthetic construct
  REGION 1..19: note = scFv domain linker
RSSTKGPKLEEGEFSEAQL

SEQ ID NO: 23 - protein, 7 aa, synthetic construct
  REGION 1..7: note = linker sequence
GGSGGRG

(19) United States
(12) Patent Application Publication
(10) Pub. No.: US 2025/0277046 A1
(43) Pub. Date: Sep. 4, 2025

(54) ANTIBODY-BASED SOLUBLE AND MEMBRANE-BOUND TWEAK MIMICKING AGONISTS WITH FCYR-INDEPENDENT ACTIVITY

(71) Applicant: JULIUS-MAXIMILIANS-UNIVERSITÄT WÜRZBURG, Würzburg (DE)

(72) Inventors: Harald Wajant, Kist (DE); Olena Zaitseva, Würzburg (DE); Christoph Otto, Würzburg (DE); Andreas Beilhack, Würzburg (DE)

(21) Appl. No.: 18/869,982

(22) PCT Filed: May 25, 2023
(86) PCT No.: PCT/EP2023/064045

§ 371 Date: Nov. 27, 2024

(30) Foreign Application Priority Data
Jun. 1, 2022 (EP) 22176698.3

(51) Int. Cl. C07K 16/28; A61K 39/00; A61P 35/00

(52) U.S. Cl. CPC C07K 16/2878; A61P 35/00; A61K 2039/505; C07K 2317/35; C07K 2317/622; C07K 2317/73; C07K 2317/75; C07K 2317/76

(57) ABSTRACT

The present invention relates to multivalent anti-Fn14 antibody constructs that show memTWEAK-mimicking agonism. The invention more specifically relates to tetra-, hexa- and octavalent antibody constructs composed of Fn14-specific Fab- and scFv domains with FcγR-independent activity. The invention also relates to methods of producing such multivalent anti-Fn14 antibody constructs, pharmaceutical compositions comprising the same, as well as their uses for treating cancer.

### FIELD OF THE INVENTION

The present invention relates to multivalent anti-Fn14 antibody constructs that show memTWEAK-mimicking agonism. The invention more specifically relates to tetra-, hexa- and octavalent antibody constructs composed of Fn14-specific Fab- and scFv domains with FcγR-independent activity. The invention also relates to methods of producing such multivalent anti-Fn14 antibody constructs, pharmaceutical compositions comprising the same, as well as their uses for treating cancer.

### BACKGROUND

Fibroblast growth factor (FGF)-inducible 14 (Fn14) is an unusual small member of the TNF receptor superfamily (TNFRSF) with an extracellular domain only comprising a single cysteine rich domain, and an intracellular tail of 28 amino acids which contains a binding site for proteins of the TNF receptor associated factor (TRAF) family (Meighan-Mantha et al., 1999; Wiley et al., 2001). Fn14 is dynamically and highly expressed during development but in healthy adult organisms Fn14 expression is largely limited to heart, ovary and mesenchymal progenitor cells (Meighan-Mantha et al., 1999; Girgenrath et al., 2010). Fn14 expression is, however, strongly upregulated in non-hematopoietic cells after tissue injury irrespective of the underlying reason (Wajant 2013). Since tumor development is inevitably associated with tissue damage and tissue remodeling, Fn14 expression is also often high in tumor cells of non-hematopoietic origin and non-transformed non-hematopoietic cells of the tumor microenvironment (Wajant 2013). The expression of Fn14 can be therefore considered as a bona fide marker for tissue remodeling and tissue injury. Fn14 signal transduction can be triggered by tumor necrosis factor (TNF)-like weak inducer of apoptosis (TWEAK), a ligand of the TNF superfamily (TNFSF) which occurs in two forms, namely as transmembrane TWEAK (memTWEAK) and as soluble TWEAK (sTWEAK) which is released from memTWEAK by proteolytic processing (Chicheportiche et al., 1997). Similar to other ligands of the TNFSF, memTWEAK and sTWEAK form homotrimeric molecules which can bind three receptor molecules (Wajant 2013). TWEAK expression has been shown for a variety of cell lines and cell types by immunohistochemistry and RT-PCR, but memTWEAK expression has doubtlessly only been demonstrated on monocytes, dendritic cells and NK cells and a very few tumor cell lines (Wajant 2013).

Importantly, sTWEAK and memTWEAK trigger different states of Fn14 activity. In response to sTWEAK, Fn14 efficiently stimulates the alternative NFκB signaling pathway and sensitizes for TNF-induced cell death (Wajant 2013). Transmembrane TWEAK triggers the same Fn14 signaling events as sTWEAK but in addition enables Fn14 to activate also the classical NFκB pathway (Wajant 2013).

Manifold and complex functions of the TWEAK/Fn14 system have been described in tissue repair and regeneration. For example, it has been demonstrated that the TWEAK/Fn14 system promotes regenerative responses after the injury of muscles, pancreas and the liver (Girgenrath et al., 2006; Wu et al., 2013; Karaca et al., 2014). However, excessive and/or chronic engagement of the TWEAK/Fn14 system can also result in tissue repair-associated adverse effects, such as fibrosis and inflammation (Kuramitsu et al. 2014; Mittal et al., 2010a,b). Thus, dependent on the context and the disease considered, both the inhibition but also the stimulation of Fn14 can elicit beneficial therapeutic effects (Wajant 2013).

The inhibition of the TWEAK/Fn14 system can be straightforwardly achieved by help of soluble Fn14-Fc fusion proteins, TWEAK neutralizing antibodies or blocking, effector function-dead Fn14 antibody variants (Wajant 2013). Specific stimulation of Fn14 signaling is, however, more challenging. Conventional sTWEAK has an extremely low serum half-life time (<20 min) (Müller et al., 2010) and oligomeric sTWEAK variants, which display memTWEAK-like activity, are modestly produced and are more challenging in translational development than antibodies. The reagents of choice to stimulate Fn14 in vivo are therefore agonistic antibodies but here arises two fundamental problem: First, although some anti-Fn14 IgG antibodies can promote to some extent in certain cell lines p100 processing, a hallmark of the alternative NFκB pathway, they are largely not agonistic and require anchoring to Fcγ receptors (FcγR) or oligomerization, e.g. by protein G or antibody crosslinking, to become fully and strongly agonistic (Salzmann et al., 2013a, Trebing, Medler). Antibody oligomerization by protein G or secondary antibodies, however, is no practicable translational option and the FcγR-binding dependent mode of anti-Fn14 agonism is inevitably associated with triggering FcγR effector function what can disturb the anticipated therapeutic effect. Second, if Fn14 antibodies become agonistic by the aforementioned means, they mimic memTWEAK, thus mimicry of sTWEAK seems hardly possible with Fn14 antibodies.

There were and are considerable preclinical efforts to target Fn14 for tumor therapy, either by exploiting Fn14 as target for antibodies with cytotoxic activity (ADCC-inducing IgG variants, antibody drug conjugates) or with blocking antibodies with the aim to interfere with protumoral Fn14 activities. Noteworthy, there are yet no attempts to target Fn14 with agonistic Fc effector function silenced antibodies to unleash the proinflammatory and cell death-enhancing activities of this receptor for tumor therapy. This is certainly not at least due to the fact that anti-Fn14 antibodies only act as effective agonists when they are presented bound to FcγR.

Thus, there remains the need for means that robustly and selectively engage Fn14 signaling without triggering unwanted FcγR-mediated activities, and that can be used in cancer therapy.

### DESCRIPTION OF THE INVENTION

The inventors have identified antibody constructs, including inter alia oligovalent variants of the Fn14-specific antibody 18D1, which mimic the activity of sTWEAK or memTWEAK independent from FcγR binding (see, for instance, FIGS. 2 and 5). Surprisingly, the antibody constructs of the invention have good antitumoral activity (see, for instance, FIG. 6). In contrast to conventional anti-Fn14 antibodies, the agonism of these novel Fn14 agonistic constructs is not limited by the availability of FcγR-expressing immune cells or competition with endogenous irrelevant antibodies for FcγR binding. It is expected that this is advantageous for therapeutic uses such as uses in the treatment of cancer. Unexpectedly, although all oligovalent anti-Fn14 constructs efficiently induced p100 processing and enhancement of TNF-induced cell death, there were considerable differences in the induction of IL8, a target of the classical NFκB pathway. Constructs only harboring multiple copies of the scFv18D1 domain largely failed to activate 118 production while constructs with similar valency but composed of Fab and scFv domains did this very efficiently (see, for instance, FIG. 2C). Thus, it is expected that the number and type of the Fn14 binding domains within an oligovalent 18D1 construct decides whether sTWEAK- or memTWEAK-like activity is mimicked. The pathway selective agonism mirrors the activities of sTWEAK and memTWEAK, the natural ligands of Fn14, but is yet without precedence for anti-Fn14 antibodies. The antibody constructs of the invention are advantageous, because they allow to selectively exert effects (e.g. pharmacological effects) similar to sTWEAK—or memTWEAK—by antibody constructs but without additional needs e.g. FcgR binding.

According to the invention, these novel potent agonistic antibody-based Fn14 constructs stimulate Fn14 with antibodies in an FcγR-independent manner, and it is expected that this advantage will translate into opening new fields of application, including clinical applications such as cancer therapy.

Accordingly, the invention relates to the following preferred embodiments:
1. A multivalent anti-Fn14 antibody construct containing at least 4 antigen binding sites for Fn14.
2. The construct of item 1, wherein the construct comprises an IgG molecule containing two of said antigen binding sites for Fn14.
3. The construct of item 1, wherein the construct comprises a modified IgG molecule, which has been modified by replacing each of the two variable domains of the heavy chain by an scFv containing one of said antigen binding sites for Fn14, and by replacing each of the two variable domains of the light chain by an scFv containing one of said antigen binding sites for Fn14.
4. The construct of item 2 or 3, wherein the construct further comprises an scFv covalently linked to the C-terminus of one of the two heavy chains of the IgG molecule, the scFv containing one of said antigen binding sites for Fn14.
5. The construct of item 4, wherein the construct further comprises an scFv covalently linked to the C-terminus of the other of the two heavy chains of the IgG molecule, the scFv containing one of said antigen binding sites for Fn14.
6. The construct of any one of items 2 to 5, wherein the construct further comprises an scFv covalently linked to the C-terminus of one of the two light chains of the IgG molecule, the scFv containing one of said antigen binding sites for Fn14.
7. The construct of item 6, wherein the construct further comprises an scFv covalently linked to the C-terminus of the other of the two light chains of the IgG molecule, the scFv containing one of said antigen binding sites for Fn14.
8. The construct of any one of items 2 to 7, wherein the IgG is IgG1, IgG2, IgG3 or IgG4.
9. The construct of any one of items 2 to 7, wherein the IgG is IgG2 or IgG4.
10. The construct according to any one of items 2 to 9, wherein the Fc domain of the IgG contains a mutation reducing FcγR binding.
11. The construct according to item 10, wherein the mutation is an N297A mutation.
12. The construct according to any one of the preceding items, wherein the construct is an Fn14 agonist.
13. The construct according to item 12, wherein the construct is capable of activating IL8 production in cells expressing Fn14, preferably in HT-1080 cells expressing Fn14.
14. The construct according to any one of the preceding items, wherein the construct contains at least 5 antigen binding sites for Fn14.
15. The construct according to any one of the preceding items, wherein the construct contains at least 6 antigen binding sites for Fn14.
16. The construct according to any one of the preceding items, wherein the construct contains 6 antigen binding sites for Fn14.
17. The construct according to any one of items 1-2, 4-5 and 8-16, wherein the construct comprises an amino acid sequence at least 85% identical to the amino acid sequence of SEQ ID NO: 2 and an amino acid sequence at least 85% identical to the amino acid sequence of SEQ ID NO: 3, preferably an amino acid sequence at least 90% identical to the amino acid sequence of SEQ ID NO: 2 and an amino acid sequence at least 90% identical to the amino acid sequence of SEQ ID NO: 3, more preferably an amino acid sequence at least 95% identical to the amino acid sequence of SEQ ID NO: 2 and an amino acid sequence at least 95% identical to the amino acid sequence of SEQ ID NO: 3, more preferably an amino acid sequence at least 98% identical to the amino acid sequence of SEQ ID NO: 2 and an amino acid sequence at least 98% identical to the amino acid sequence of SEQ ID NO: 3, more preferably an amino acid sequence at least 99% identical to the amino acid sequence of SEQ ID NO: 2 and an amino acid sequence at least 99% identical to the amino acid sequence of SEQ ID NO: 3, and most preferably the amino acid sequence of SEQ ID NO: 2 and the amino acid sequence of SEQ ID NO: 3.
18. The construct according to any one of items 1-2 and 6-16, wherein the construct comprises an amino acid sequence at least 85% identical to the amino acid sequence of SEQ ID NO: 1 and an amino acid sequence at least 85% identical to the amino acid sequence of SEQ ID NO: 4, preferably an amino acid sequence at least 90% identical to the amino acid sequence of SEQ ID NO: 1 and an amino acid sequence at least 90% identical to the amino acid sequence of SEQ ID NO: 4, more preferably an amino acid sequence at least 95% identical to the amino acid sequence of SEQ ID NO: 1 and an amino acid sequence at least 95% identical to the amino acid sequence of SEQ ID NO: 4, more preferably an amino acid sequence at least 98% identical to the amino acid sequence of SEQ ID NO: 1 and an amino acid sequence at least 98% identical to the amino acid sequence of SEQ ID NO: 4, more preferably an amino acid sequence at least 99% identical to the amino acid sequence of SEQ ID NO: 1 and an amino acid sequence at least 99% identical to the amino acid sequence of SEQ ID NO: 4, and most preferably the amino acid sequence of SEQ ID NO: 1 and the amino acid sequence of SEQ ID NO: 4.
19. The construct according to any one of items 1-2 and 4-16, wherein the construct comprises an amino acid sequence at least 85% identical to the amino acid sequence of SEQ ID NO: 2 and an amino acid sequence at least 85% identical to the amino acid sequence of SEQ ID NO: 4, preferably an amino acid sequence at least 90% identical to the amino acid sequence of SEQ ID NO: 2 and an amino acid sequence at least 90% identical to the amino acid sequence of SEQ ID NO: 4, more preferably an amino acid sequence at least 95% identical to the amino acid sequence of SEQ ID NO: 2 and an amino acid sequence at least 95% identical to the amino acid sequence of SEQ ID NO: 4, more preferably an amino acid sequence at least 98% identical to the amino acid sequence of SEQ ID NO: 2 and an amino acid sequence at least 98% identical to the amino acid sequence of SEQ ID NO: 4, more preferably an amino acid sequence at least 99% identical to the amino acid sequence of SEQ ID NO: 2 and an amino acid sequence at least 99% identical to the amino acid sequence of SEQ ID NO: 4, and most preferably the amino acid sequence of SEQ ID NO: 2 and the amino acid sequence of SEQ ID NO: 4.
20. The construct according to any one of items 1, 3 and 8-16, wherein the construct comprises an amino acid sequence at least 85% identical to the amino acid sequence of SEQ ID NO: 6 and an amino acid sequence at least 85% identical to the amino acid sequence of SEQ ID NO: 5, preferably an amino acid sequence at least 90% identical to the amino acid sequence of SEQ ID NO: 6 and an amino acid sequence at least 90% identical to the amino acid sequence of SEQ ID NO: 5, more preferably an amino acid sequence at least 95% identical to the amino acid sequence of SEQ ID NO: 6 and an amino acid sequence at least 95% identical to the amino acid sequence of SEQ ID NO: 5, more preferably an amino acid sequence at least 98% identical to the amino acid sequence of SEQ ID NO: 6 and an amino acid sequence at least 98% identical to the amino acid sequence of SEQ ID NO: 5, more preferably an amino acid sequence at least 99% identical to the amino acid sequence of SEQ ID NO: 6 and an amino acid sequence at least 99% identical to the amino acid sequence of SEQ ID NO: 5, and most preferably the amino acid sequence of SEQ ID NO: 6 and the amino acid sequence of SEQ ID NO: 5.
21. The construct according to any one of items 1, 3-5 and 8-16, wherein the construct comprises an amino acid sequence at least 85% identical to the amino acid sequence of SEQ ID NO: 8 and an amino acid sequence at least 85% identical to the amino acid sequence of SEQ ID NO: 5, preferably an amino acid sequence at least 90% identical to the amino acid sequence of SEQ ID NO: 8 and an amino acid sequence at least 90% identical to the amino acid sequence of SEQ ID NO: 5, more preferably an amino acid sequence at least 95% identical to the amino acid sequence of SEQ ID NO: 8 and an amino acid sequence at least 95% identical to the amino acid sequence of SEQ ID NO: 5, more preferably an amino acid sequence at least 98% identical to the amino acid sequence of SEQ ID NO: 8 and an amino acid sequence at least 98% identical to the amino acid sequence of SEQ ID NO: 5, more preferably an amino acid sequence at least 99% identical to the amino acid sequence of SEQ ID NO: 8 and an amino acid sequence at least 99% identical to the amino acid sequence of SEQ ID NO: 5, and most preferably the amino acid sequence of SEQ ID NO: 8 and the amino acid sequence of SEQ ID NO: 5.
22. The construct according to any one of items 1, 3 and 6-16, wherein the construct comprises an amino acid sequence at least 85% identical to the amino acid sequence of SEQ ID NO: 6 and an amino acid sequence at least 85% identical to the amino acid sequence of SEQ ID NO: 7, preferably an amino acid sequence at least 90% identical to the amino acid sequence of SEQ ID NO: 6 and an amino acid sequence at least 90% identical to the amino acid sequence of SEQ ID NO: 7, more preferably an amino acid sequence at least 95% identical to the amino acid sequence of SEQ ID NO: 6 and an amino acid sequence at least 95% identical to the amino acid sequence of SEQ ID NO: 7, more preferably an amino acid sequence at least 98% identical to the amino acid sequence of SEQ ID NO: 6 and an amino acid sequence at least 98% identical to the amino acid sequence of SEQ ID NO: 7, more preferably an amino acid sequence at least 99% identical to the amino acid sequence of SEQ ID NO: 6 and an amino acid sequence at least 99% identical to the amino acid sequence of SEQ ID NO: 7, and most preferably the amino acid sequence of SEQ ID NO: 6 and the amino acid sequence of SEQ ID NO: 7.
23. The construct according to any one of items 1 and 3-16, wherein the construct comprises an amino acid sequence at least 85% identical to the amino acid sequence of SEQ ID NO: 8 and an amino acid sequence at least 85% identical to the amino acid sequence of SEQ ID NO: 7, preferably an amino acid sequence at least 90% identical to the amino acid sequence of SEQ ID NO: 8 and an amino acid sequence at least 90% identical to the amino acid sequence of SEQ ID NO: 7, more preferably an amino acid sequence at least 95% identical to the amino acid sequence of SEQ ID NO: 8 and an amino acid sequence at least 95% identical to the amino acid sequence of SEQ ID NO: 7, more preferably an amino acid sequence at least 98% identical to the amino acid sequence of SEQ ID NO: 8 and an amino acid sequence at least 98% identical to the amino acid sequence of SEQ ID NO: 7, more preferably an amino acid sequence at least 99% identical to the amino acid sequence of SEQ ID NO: 8 and an amino acid sequence at least 99% identical to the amino acid sequence of SEQ ID NO: 7, and most preferably the amino acid sequence of SEQ ID NO: 8 and the amino acid sequence of SEQ ID NO: 7.
24. The construct according to any one of items 1-2, 4-5 and 8-16, wherein the construct comprises an amino acid sequence at least 85% identical to the amino acid sequence of SEQ ID NO: 10 and an amino acid sequence at least 85% identical to the amino acid sequence of SEQ ID NO: 11, preferably comprises an amino acid sequence at least 90% identical to the amino acid sequence of SEQ ID NO: 10 and an amino acid sequence at least 90% identical to the amino acid sequence of SEQ ID NO: 11, more preferably comprises an amino acid sequence at least 95% identical to the amino acid sequence of SEQ ID NO: 10 and an amino acid sequence at least 95% identical to the amino acid sequence of SEQ ID NO: 11, more preferably an amino acid sequence at least 98% identical to the amino acid sequence of SEQ ID NO: 10 and an amino acid sequence at least 98% identical to the amino acid sequence of SEQ ID NO: 11, more preferably an amino acid sequence at least 99% identical to the amino acid sequence of SEQ ID NO: 10 and an amino acid sequence at least 99% identical to the amino acid sequence of SEQ ID NO: 11, and most preferably the amino acid sequence of SEQ ID NO: 10 and the amino acid sequence of SEQ ID NO: 11.
25. The construct according to any one of items 1-2 and 6-16, wherein the construct comprises an amino acid sequence at least 85% identical to the amino acid sequence of SEQ ID NO: 9 and an amino acid sequence at least 85% identical to the amino acid sequence of SEQ ID NO: 12, preferably an amino acid sequence at least 90% identical to the amino acid sequence of SEQ ID NO: 9 and an amino acid sequence at least 90% identical to the amino acid sequence of SEQ ID NO: 12, more preferably an amino acid sequence at least 95% identical to the amino acid sequence of SEQ ID NO: 9 and an amino acid sequence at least 95% identical to the amino acid sequence of SEQ ID NO: 12, more preferably an amino acid sequence at least 98% identical to the amino acid sequence of SEQ ID NO: 9 and an amino acid sequence at least 98% identical to the amino acid sequence of SEQ ID NO: 12, more preferably an amino acid sequence at least 99% identical to the amino acid sequence of SEQ ID NO: 9 and an amino acid sequence at least 99% identical to the amino acid sequence of SEQ ID NO: 12, and most preferably the amino acid sequence of SEQ ID NO: 9 and the amino acid sequence of SEQ ID NO: 12.
26. The construct according to any one of items 1-2 and 4-16, wherein the construct comprises an amino acid sequence at least 85% identical to the amino acid sequence of SEQ ID NO: 10 and an amino acid sequence at least 85% identical to the amino acid sequence of SEQ ID NO: 12, preferably an amino acid sequence at least 90% identical to the amino acid sequence of SEQ ID NO: 10 and an amino acid sequence at least 90% identical to the amino acid sequence of SEQ ID NO: 12, more preferably an amino acid sequence at least 95% identical to the amino acid sequence of SEQ ID NO: 10 and an amino acid sequence at least 95% identical to the amino acid sequence of SEQ ID NO: 12, more preferably an amino acid sequence at least 98% identical to the amino acid sequence of SEQ ID NO: 10 and an amino acid sequence at least 98% identical to the amino acid sequence of SEQ ID NO: 12, more preferably an amino acid sequence at least 99% identical to the amino acid sequence of SEQ ID NO: 10 and an amino acid sequence at least 99% identical to the amino acid sequence of SEQ ID NO: 12, and most preferably the amino acid sequence of SEQ ID NO: 10 and the amino acid sequence of SEQ ID NO: 12.
27. The construct according to any one of items 1, 3 and 8-16, wherein the construct comprises an amino acid sequence at least 85% identical to the amino acid sequence of SEQ ID NO: 14 and an amino acid sequence at least 85% identical to the amino acid sequence of SEQ ID NO: 13, preferably an amino acid sequence at least 90% identical to the amino acid sequence of SEQ ID NO: 14 and an amino acid sequence at least 90% identical to the amino acid sequence of SEQ ID NO: 13, more preferably an amino acid sequence at least 95% identical to the amino acid sequence of SEQ ID NO: 14 and an amino acid sequence at least 95% identical to the amino acid sequence of SEQ ID NO: 13, more preferably an amino acid sequence at least 98% identical to the amino acid sequence of SEQ ID NO: 14 and an amino acid sequence at least 98% identical to the amino acid sequence of SEQ ID NO: 13, more preferably an amino acid sequence at least 99% identical to the amino acid sequence of SEQ ID NO: 14 and an amino acid sequence at least 99% identical to the amino acid sequence of SEQ ID NO: 13, and most preferably the amino acid sequence of SEQ ID NO: 14 and the amino acid sequence of SEQ ID NO: 13.
28. The construct according to any one of the preceding items, wherein the construct comprises a trimerization domain.
29. The construct according to item 28, wherein the trimerization domain is a trimerization domain of tenascin C.
30. The construct according to any one of items 28 and 29, wherein the trimerization domain comprises the amino acid sequence of SEQ ID NO: 21 or a sequence at least 70% identical thereto, preferably at least 80% identical thereto, more preferably at least 85% identical thereto, more preferably at least 90% identical thereto, more preferably at least 93% identical thereto, and more preferably at least 96% identical thereto.
31. The construct according to any one of items 28 to 30, wherein the construct is a construct according to any one of items 2-3, and wherein the trimerization domain is linked to the C-terminus of the constant domains of the heavy chains of the IgG.
32. The construct according to any one of items 1, 2, 8, 10-16 and 28-31, wherein the construct comprises an amino acid sequence at least 85% identical to the amino acid sequence of SEQ ID NO: 15 and an amino acid sequence at least 85% identical to the amino acid sequence of SEQ ID NO: 3, preferably an amino acid sequence at least 90% identical to the amino acid sequence of SEQ ID NO: 15 and an amino acid sequence at least 90% identical to the amino acid sequence of SEQ ID NO: 3, more preferably an amino acid sequence at least 95% identical to the amino acid sequence of SEQ ID NO: 15 and an amino acid sequence at least 95% identical to the amino acid sequence of SEQ ID NO: 3, more preferably an amino acid sequence at least 98% identical to the amino acid sequence of SEQ ID NO: 15 and an amino acid sequence at least 98% identical to the amino acid sequence of SEQ ID NO: 3, more preferably an amino acid sequence at least 99% identical to the amino acid sequence of SEQ ID NO: 15 and an amino acid sequence at least 99% identical to the amino acid sequence of SEQ ID NO: 3, and most preferably the amino acid sequence of SEQ ID NO: 15 and the amino acid sequence of SEQ ID NO: 3.
33. The construct according to any one of items 1, 2, 8, and 10-16, wherein the construct comprises an amino acid sequence at least 85% identical to the amino acid sequence of SEQ ID NO: 16 and an amino acid sequence at least 85% identical to the amino acid sequence of SEQ ID NO: 3, preferably an amino acid sequence at least 90% identical to the amino acid sequence of SEQ ID NO: 16 and an amino acid sequence at least 90% identical to the amino acid sequence of SEQ ID NO: 3, more preferably an amino acid sequence at least 95% identical to the amino acid sequence of SEQ ID NO: 16 and an amino acid sequence at least 95% identical to the amino acid sequence of SEQ ID NO: 3, more preferably an amino acid sequence at least 98% identical to the amino acid sequence of SEQ ID NO: 16 and an amino acid sequence at least 98% identical to the amino acid sequence of SEQ ID NO: 3, more preferably an amino acid sequence at least 99% identical to the amino acid sequence of SEQ ID NO: 16 and an amino acid sequence at least 99% identical to the amino acid sequence of SEQ ID NO: 3, and most preferably the amino acid sequence of SEQ ID NO: 16 and the amino acid sequence of SEQ ID NO: 3.
34. The construct according to any one of items 1, 3, 8, 10-16 and 28-31, wherein the construct comprises an amino acid sequence at least 85% identical to the amino acid sequence of SEQ ID NO: 17 and an amino acid sequence at least 85% identical to the amino acid sequence of SEQ ID NO: 5, preferably an amino acid sequence at least 90% identical to the amino acid sequence of SEQ ID NO: 17 and an amino acid sequence at least 90% identical to the amino acid sequence of SEQ ID NO: 5, more preferably an amino acid sequence at least 95% identical to the amino acid sequence of SEQ ID NO: 17 and an amino acid sequence at least 95% identical to the amino acid sequence of SEQ ID NO: 5, more preferably an amino acid sequence at least 98% identical to the amino acid sequence of SEQ ID NO: 17 and an amino acid sequence at least 98% identical to the amino acid sequence of SEQ ID NO: 5, more preferably an amino acid sequence at least 99% identical to the amino acid sequence of SEQ ID NO: 17 and an amino acid sequence at least 99% identical to the amino acid sequence of SEQ ID NO: 5, and most preferably the amino acid sequence of SEQ ID NO: 17 and the amino acid sequence of SEQ ID NO: 5.
35. The construct according to any one of items 1, 3, 8-16 and 28-30, wherein the construct comprises an amino acid sequence at least 85% identical to the amino acid sequence of SEQ ID NO: 18, preferably an amino acid sequence at least 90% identical to the amino acid sequence of SEQ ID NO: 18, more preferably an amino acid sequence at least 95% identical to the amino acid sequence of SEQ ID NO: 18, more preferably an amino acid sequence at least 98% identical to the amino acid sequence of SEQ ID NO: 18, more preferably an amino acid sequence at least 99% identical to the amino acid sequence of SEQ ID NO: 18, and most preferably the amino acid sequence of SEQ ID NO: 18.
36. The construct according to any one of the preceding items, further comprising an N-terminal amino acid sequence comprising one or two of the following ((a) and (b)): (a) a leader sequence comprising the amino acid sequence of SEQ ID NO: 19, optionally followed by a first linker sequence, and (b) a flag tag comprising the amino acid sequence of SEQ ID NO: 20, optionally followed by a second linker sequence.
37. A pharmaceutical composition comprising the construct according to any one of the preceding items.
38. A pharmaceutical composition according to item 37 or a construct according to any one of items 1 to 36, for use in the treatment of cancer.
39. The pharmaceutical composition or construct for use according to item 38, wherein the cancer is a cancer expressing Fn14.
40. The pharmaceutical composition or construct for use according to any one of items 38 to 39, wherein the cancer is a solid cancer.
41. A nucleic acid, or a set of nucleic acids, encoding the construct of any one of items 1-36.
42. A recombinant cell containing a nucleic acid, or a set of nucleic acids, according to item 41 and expressing the construct of any one of items 1-36.
43. A method for producing a construct of any one of items 1-36, the method comprising expressing said construct in a recombinant cell according to item 42 from the nucleic acid or set of nucleic acids according to item 41, wherein the method optionally further comprises purifying and formulating said construct into a pharmaceutical composition according to item 37.

### BRIEF DESCRIPTION OF THE DRAWINGS

FIG. 1: Antibody variants of the anti-Fn14-antibody 18D1. (A) Scheme of the domain architecture of the various 18D1 variants. (B) Western blot analysis of 10 μl supernatant of HEK293 cells transiently expressing the proteins shown in (A).

FIG. 2: Triggering of Fn14 signaling by oligovalent 18D1 constructs. (A) HeLa-RIPK3-FADDKO cells were treated with 1 ng/ml TNF which alone induces no or only modest cell death. Cells were treated in addition with cell culture supernatants containing the indicated 18D1 constructs. Cotreatment with (200 ng/ml) Flag-sTWEAK was performed to define maximum TNF killing in TWEAK-sensitized cells indicated by the dotted line. (B) Kym-1 cells were treated overnight with cell culture supernatants containing the various 18D1 constructs or with Flag-sTWEAK. To prevent cell death induction 20 μM ZVAD were added. Total cell lysates were analyzed by western blotting with respect to p100 processing and expression of the alternative NFκB pathway target TRAF1. (C) HT1080 cells were treated with the various 18D1 constructs or with anti-Flag M2 oligomerized Flag-sTWEAK overnight. IL8 concentrations were evaluate by ELISA.

FIG. 3: 18D1-(5) to 18D1-(8) inhibit memTWEAK-induced classical NFκB signaling pathway-mediated IL8 expression. (A, B) HT1080 cells were pretreated with 30 μg/ml of the indicated 18D1 variants and were then challenged overnight with memTWEAK and, as a negative control, empty vector (EV) transfected HEK293 cells (A) or with 500 ng/ml Fc-sTWEAK (B). Shown are the average of 7 (A) and 4 (B) independent experiments. (C) HT1080 cells were stimulated with anti-Flag antibody M2 (0.5 μg/ml) oligomerized Flag-sTWEAK (200 ng/ml) or 1 μg/ml 18D1-(4) in the presence and absence of the IKK2 inhibitor TPCA-1 (20 μM). Next day, IL8 production was again determined by ELISA. (D) Kym-1 cells were stimulated with 200 ng/ml Flag-sTWEAK (200 ng/ml) or 1 μg/ml 18D1-(4) in the presence and absence of TPCA-1 (20 μM) and the next day, p100 processing and TRAF1 induction were assayed by western blotting.

FIG. 4: Purification and analysis of tetra- and hexavalent anti-Fn14 18D1 variants. (A) Purified proteins were separated by SDS-PAGE and visualized by silver staining. (B) Gel filtration analysis of purified proteins. (C-E) Analysis of the ability of purified proteins from part B to trigger enhancement of TNF-induced toxicity (C), IL8 production (D) and p100 processing (E).

FIG. 5: Triggering of Fn14 signaling by PDL192 type (2) and type (4) constructs. (A-C) The activity of PDL192-(2) and PDL192-(4) were analyzed as in FIG. 2 with respect to enhancement of TNF-induced cell death in HeLa-RIPK3-FADDKO cells (A), stimulation of p100 processing and TRAF1 induction (B) and upregulation of IL8 production (C). (D, E) Purified PDL192-(2) and PDL192-(4) along with the parental antibody PDL192 were analyzed by SDS-PAGE and silver staining (D) or gel filtration analysis (E).

FIG. 6: Antitumoral activity of 18D1-(2). Mouse tumor organoids (MTOs), derived of tumors originated from mice with Apc^{ko/ko}, Kras^{LSL-G12D}, Tgfbr2^{ko/ko} and Trp53^{ko/ko} intestinal stem cells (Tauriello et al., 2018), were expanded in vitro and injected subcutaneously into the flank of syngeneic C57BL/6J recipients. Two weeks after tumors have been initially established, mice were treated three times per week for two weeks with 200 μg of 18D1-(2). For comparison mice were treated with MSA-sTWEAK, a fusion protein of sTWEAK with serum albumin and thus prolonged serum retention, and Fc (DANA)-sTWEAK. Tumor size (A) and tumor weight (B) were significantly reduced in mice treated with 18D1-(2) and there was also significantly reduced tumor weight after Fc (DANA)-sTWEAK treatment and a trend to lower tumor volume. MSA-sTWEAK treated mice reached no significant antitumoral effect.

FIG. 7: Triggering of Fn14 signaling by oligovalent 18D1 constructs. HT1080 cells were treated with the various 18D1 constructs or with 18D1 constructs crosslinked with G protein. IL8 concentrations were measured. (A) Results for the constructs 18D1-(1) to 18D1-(4). (B) Results for the constructs 18D1-(5) to 18D1-(8).

FIG. 8: soluble TWEAK also inhibits the memTWEAK-induced IL8 response. HT1080 cells, which produce IL8 in response to cells expressing membraneTWEAK, were mixed with HEK293 cells transiently transfected with empty vector (EV, negative control) or an expression plasmid encoding a non-cleavable form of membrane TWEAK (nc-memTWEAK). Cell mixtures were further supplemented with the indicated concentration of soluble TWEAK (sTWEAK). Next day, IL-8 production was evaluated by ELISA.

FIG. 9: Further constructs based on the anti-Fn14-antibody 18D1. N297A refers to the corresponding mutation in the heavy chain of human IgG1 reducing FcgR Interaction. N-RGY refers to the N297A mutation plus mutations promoting IgG1 hexamerization. Mutations promoting IgG1 hexamerization have been described in “A Novel Platform for the Potentiation of Therapeutic Antibodies Based on Antigen-Dependent Formation of IgG Hexamers at the Cell Surface.” (de Jong R N, Beurskens F J, Verploegen S, Strumane K, van Kampen M D, Voorhorst M, Horstman W, Engelberts P J, Oostindie S C, Wang G, Heck A J, Schuurman J, Parren P W. PLOS Biol. 2016 Jan. 6; 14 (1): e1002344.).

### DETAILED DESCRIPTION OF THE INVENTION

### Definitions and General Techniques

Unless otherwise defined below, the terms used in the present invention shall be understood in accordance with their common meaning known to the person skilled in the art. All publications, patents and patent applications cited herein are hereby incorporated by reference in their entirety for all purposes. Publications referred to herein may be cited by specifying the full literature reference in the text, or by naming the author and the publication year (e.g., “Kuramitsu et al. 2014”) and by specifying the corresponding full literature reference in the “references” section.

All proteins in accordance with the invention, including the antibody constructs of the invention, can be obtained by methods known in the art. Such methods include methods for the production of recombinant proteins. The antibody constructs of the invention can be expressed in recombinant host cells according to the invention. Recombinant host cells of the invention are preferably mammalian cells such as CHO and HEK cells. It will be understood that the antibody constructs of the invention are meant to optionally include a secretion signal peptide sequence. Similarly, the antibody constructs of the invention are meant to also optionally include affinity tags, e.g. in order to facilitate purification, and optional protease cleavage sites between the tag and the antibody construct, e.g. in order to facilitate removal of the tags by protease cleavage. Likewise, it will be understood that the antibody constructs of the invention are meant to optionally include the respective pro-peptides.

It is also understood that any reference to amino acid sequences referred to herein is meant to encompass not only the unmodified amino acid sequence but also typical posttranslational modifications of these amino acid sequences (e.g., glycosylation or deamidation of amino acids, the clipping of particular amino acids or other posttranslational modifications) occurring in cellular expression systems known in the art, including insect cells as well as mammalian cells such as CHO and HEK cells.

The term “antibody” as used herein refers to any functional antibody that is capable of specific binding to the antigen of interest, as generally outlined in chapter 7 of Paul, W. E. (Ed.).: Fundamental Immunology 2nd Ed. Raven Press, Ltd., New York 1989, which is incorporated herein by reference. Without particular limitation, the term “antibody” encompasses antibodies from any appropriate source species, including chicken and mammalian such as mouse, goat, non-human primate and human. Preferably, the antibody is a humanized or human antibody. The antibody is preferably a monoclonal antibody which can be prepared by methods well-known in the art. The term “antibody” encompasses an IgG-1, -2, -3, or -4, IgE, IgA, IgM, or IgD isotype antibody. The term “antibody” encompasses monomeric antibodies (such as IgD, IgE, IgG) or oligomeric antibodies (such as IgA or IgM). The term “antibody” also encompasses—without particular limitations—isolated antibodies and modified antibodies such as genetically engineered antibodies, e.g. chimeric, humanized or human antibodies. For example, the terms “IgG1”, “IgG2”, “IgG3” or “IgG4” as used in accordance with the invention encompass chimeric, humanized and human IgG1, IgG2, IgG3 and IgG4, respectively.

As used herein, an “IgG” can be a naturally occurring IgG or a mutated IgG, as is known in the art.

For IgG1 such as chimeric, humanized or human IgG1, known mutations include, but are not limited to: N297A, N297D, N297Q, N297G, E233P, L234A, L234F, L235A, L235E, P331S, P329A, P329G, P331S, P238S, LALA (LALA=mutation of Leucine 234 to Alanine and Leucine 235 to Alanine), A330S, G237A, M252Y, S254T, S228P, T256E, M252F, M252S, M252W, M252T, T256S, T256R, T256Q, T256D, H268A, and combinations thereof. Known IgG1 mutations also include E345K or E430G which promote IgG1 hexamerization.

For IgG2 such as chimeric, humanized or human IgG2, known mutations include, but are not limited to: V234A, G237A, P238S, H268A, H268Q, A330S, P331S, P233S, V309L, and combinations thereof.

For IgG3 such as chimeric, humanized or human IgG3, known mutations include, but are not limited to: replacement of the CH2 domain of IgG3 with the CH2 domain of IgG2, removal of Fc glycosylation, mutations of L322, L276, N297, and combinations thereof.

For IgG4 such as chimeric, humanized or human IgG4, known mutations include, but are not limited to: V234A, L235E, LALA (Mutation of Leucine 234 to Alanine and Leucine 235 to Alanine), F234A, P329G, S228P, G237A, P238S, G236 deletion, and combinations thereof.

The nomenclature of the antibodies, antibody fragments and mutations thereof follows the terms as known in the art. It will be understood by a person skilled in the art that in accordance with the invention, this known nomenclature is to be applied also to the antibody constructs of the invention. For example, the known nomenclature is to be applied also to an antibody which forms part of an antibody construct of the invention. For instance, where terms relating antibody mutations such as “N297A” are used herein in connection with an IgG1 which forms part of an antibody construct of the invention, it will be understood that these terms are based on known antibody nomenclature.

Each monomer of an antibody comprises two heavy chains and two light chains, as generally known in the art. Of these, each heavy and light chain comprises a variable domain (termed VH for the heavy chain and VL for the light chain) which is important for antigen binding. These heavy and light chain variable domains comprise (in an N-terminal to C-terminal order) the regions FR1, CDR1, FR2, CDR2, FR3, CDR3, and FR4 (FR, framework region; CDR, complementarity determining region which is also known as hypervariable region). The identification and assignment of the above-mentioned antibody regions within the antibody sequence is generally in accordance with Kabat et al. (Sequences of proteins of immunological interest, U.S. Dept. of Health and Human Services, Public Health Service, National Institutes of Health, Bethesda, Md. 1983), or Chothia et al. (Conformations of immunoglobulin hypervariable regions. Nature. 1989 Dec. 21-28; 342 (6252): 877-83.), or may be performed by using the IMGT/V-QUEST software described in Giudicelli et al. (IMGT/V-QUEST, an integrated software program for immunoglobulin and T cell receptor V-J and V-D-J rearrangement analysis. Nucleic Acids Res. 2004 Jul. 1; 32 (Web Server issue): W435-40.), which is incorporated herein by reference. Preferably, the antibody regions indicated above are identified and assigned by using the IMGT/V-QUEST software.

A “monoclonal antibody” is an antibody from an essentially homogenous population of antibodies, wherein the antibodies are substantially identical in sequence (i.e. identical except for minor fraction of antibodies containing naturally occurring sequence modifications such as amino acid modifications at their N- and C-termini). Unlike polyclonal antibodies which contain a mixture of different antibodies directed to either a single epitope or to numerous different epitopes, monoclonal antibodies are directed to the same epitope and are therefore highly specific. The term “monoclonal antibody” includes (but is not limited to) antibodies which are obtained from a monoclonal cell population derived from a single cell clone, as for instance the antibodies generated by the hybridoma method described in Köhler and Milstein (Nature, 1975 Aug. 7; 256 (5517): 495-7) or Harlow and Lane (“Antibodies: A Laboratory Manual” Cold Spring Harbor Laboratory Press, Cold Spring Harbor, New York 1988). A monoclonal antibody may also be obtained from other suitable methods, including phage display techniques such as those described in Clackson et al. (Nature. 1991 Aug. 15; 352 (6336): 624-8) or Marks et al. (J Mol Biol. 1991 Dec. 5; 222 (3): 581-97). A monoclonal antibody may be an antibody that has been optimized for antigen-binding properties such as decreased Kd values, optimized association and dissociation kinetics by methods known in the art. For instance, Kd values may be optimized by display methods including phage display, resulting in affinity-matured monoclonal antibodies. The term “monoclonal antibody” is not limited to antibody sequences from particular species of origin or from one single species of origin. Thus, the meaning of the term “monoclonal antibody” encompasses chimeric monoclonal antibodies such as humanized monoclonal antibodies and human antibodies.

“Humanized antibodies” are antibodies which contain human sequences and a minor portion of non-human sequences which confer binding specificity to an antigen of interest. Typically, humanized antibodies are generated by replacing hypervariable region sequences from a human acceptor antibody by hypervariable region sequences from a non-human donor antibody (e.g. a mouse, rabbit, rat donor antibody) that binds to an antigen of interest. In some cases, framework region sequences of the acceptor antibody may also be replaced by the corresponding sequences of the donor antibody. In addition to the sequences derived from the donor and acceptor antibodies, a “humanized antibody” may either contain other (additional or substitute) residues or sequences or not. Such other residues or sequences may serve to further improve antibody properties such as binding properties (e.g. to decrease Kd values) and/or immunogenic properties (e.g. to decrease antigenicity in humans). Non-limiting examples for methods to generate humanized antibodies are known in the art, e.g. from Riechmann et al. (Nature. 1988 Mar. 24; 332 (6162): 323-7) or Jones et al. (Nature. 1986 May 29-June 4; 321 (6069): 522-5).

The term “human antibody” relates to an antibody containing human variable and constant domain sequences. This definition encompasses antibodies having human sequences bearing single amino acid substitutions or modifications which may serve to further improve antibody properties such as binding properties (e.g. to decrease Kd values) and/or immunogenic properties (e.g. to decrease antigenicity in humans). The term “human antibody” excludes humanized antibodies where a portion of non-human sequences confers binding specificity to an antigen of interest.

An “antigen-binding portion” of an antibody as used herein refers to a portion of an antibody that retains the capability of the antibody to specifically bind to the antigen. This capability can, for instance, be determined by determining the capability of the antigen-binding portion to compete with the antibody for specific binding to the antigen by methods known in the art. The antigen-binding portion may contain one or more fragments of the antibody. Without particular limitation, the antigen-binding portion can be produced by any suitable method known in the art, including recombinant DNA methods and preparation by chemical or enzymatic fragmentation of antibodies. Antigen-binding portions may be Fab fragments, F(ab′) fragments, Fab2 fragments, single-chain variable fragments (scFv), single-domain antibodies, diabodies or any other portion(s) of the antibody that retain the capability of the antibody to specifically bind to the antigen. It will be understood that in accordance with the meaning of the term “Fab2” as known in the art, the term “Fab2” is synonymous with the terms “Fab2”, “Fab2”, and “FAB2”. Thus, for the purposes of the present application, the term “Fab2” is used interchangeably with the terms “Fab2”, “Fab2”, and “FAB2”.

An “antibody construct” according to the invention contains at least 4 antigen binding sites for Fn14, as defined herein, and is a protein. An antibody construct is capable of binding to its specific target antigen, i.e. Fn14. It is understood that an antibody construct according to the invention may be a protein consisting of a single polypeptide chain, or it may be a protein wherein two or more polypeptide chains are linked together. They can be linked together covalently, e.g., by covalent linkages. Such covalent linkages may be one or more disulfide linkages. Alternatively, the covalent linkages may be obtained by chemical conjugation (preferably by chemical conjugation using click chemistry), and/or they may be any other covalent linkage which is known in the art as a suitable link for proteins. Alternatively, an antibody construct according to the invention may be a protein wherein two or more polypeptide chains are linked together non-covalently (e.g., by non-covalent interactions). For example, the IgG1 mutations E345K and/or E430G promote IgG1 hexamerization. According to the invention, such IgG1 mutations can be included in the antibody constructs of the invention, in order to obtain hexameric antibody constructs.

Generally, in connection with all antibody constructs of the invention, it will be understood that the parts of the antibody constructs may be fused using linker sequences. In that case, the antibody construct of the invention will comprise such linker sequences. Suitable linker sequences are known in the art and comprise, for example, peptide linkers, without being limited thereto.

An “antibody construct” according to the invention may an antibody construct which is derivatized or linked to a different molecule. For example, molecules that may be linked to the antibody construct are a molecular label (e.g. a fluorescent, luminescent, colored or radioactive molecule) and/or a pharmaceutical agent.

The meaning of the terms “antigen binding site” and “antigen binding sites” as used herein is known in the art. Typically, an antigen binding site comprises six complementarity-determining regions (CDRs). The six complementarity-determining regions (CDRs) are typically located in the VH and VL domains, i.e. a CDR1, a CDR2 and a CDR3 in the VH (i.e., in the variable domain of the heavy chain) and a CDR1, a CDR2 and a CDR3 in the VL (i.e., in the variable domain of the light chain). Various forms of antigen binding sites are known in the art and include, without limitation, antigen binding sites contained in the VH and VH of antibodies, or antigen binding sites contained in their antibody fragments such as scFvs.

The terms “Fc gamma receptor” or “FcγR” as used herein refer to any member of the family of proteins that bind the IgG antibody Fc domain and are encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2). An FcγR can be from any organism, including but not limited to humans, mice, rats, rabbits, and monkeys, and is preferably from humans. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

As used herein, the term “mutation reducing FcγR binding” in connection with the Fc domain of an IgG of the antibody constructs of the invention encompasses any mutation that reduces binding of the antibody construct to at least one of the Fc gamma receptors as compared to a corresponding reference antibody construct where the mutation is absent from the Fc domain. Whether a mutation reduces FcγR binding to an Fc gamma receptor can be determined by methods known in the art such as comparative surface plasmon resonance measurements of the binding of the antibody construct and the reference antibody construct to the respective immobilized recombinant Fc gamma receptor.

Mutations reducing FcγR binding are well known in the art.

For IgG1 such as chimeric, humanized or human IgG1, known mutations reducing FcγR binding include, but are not limited to: N297A, N297D, N297Q, N297G, E233P, L234A, L234F, L235A, L235E, P331S, P329A, P329G, P331S, P238S, LALA (LALA=mutation of Leucine 234 to Alanine and Leucine 235 to Alanine), A330S, G237A, M252Y, S254T, S228P, T256E, M252F, M252S, M252W, M252T, T256S, T256R, T256Q, T256D, H268A, and combinations thereof.

For IgG2 such as chimeric, humanized or human IgG2, known mutations reducing FcγR binding include, but are not limited to: V234A, G237A, P238S, H268A, H268Q, A330S, P331S, P233S, V309L, and combinations thereof.

For IgG3 such as chimeric, humanized or human IgG3, known mutations reducing FcγR binding include, but are not limited to: replacement of the CH2 domain of IgG3 with the CH2 domain of IgG2, removal of Fc glycosylation, mutations of L322, L276, N297, and combinations thereof.

For IgG4 such as chimeric, humanized or human IgG4, known mutations reducing FcγR binding include, but are not limited to: V234A, L235E, LALA (Mutation of Leucine 234 to Alanine and Leucine 235 to Alanine), F234A, P329G, S228P, G237A, P238S, G236 deletion, and combinations thereof.

The term “Fn14 Agonist” refers to a molecule that is able to trigger Fn14 signal transduction. Fn14 signal transduction can be triggered by tumor necrosis factor (TNF)-like weak inducer of apoptosis (TWEAK), a ligand of the TNF superfamily (TNFSF) which occurs in two forms, namely as transmembrane TWEAK (memTWEAK) and as soluble TWEAK (sTWEAK) which is released from memTWEAK by proteolytic processing (Chicheportiche et al., 1997). sTWEAK and memTWEAK trigger different states of Fn14 activity. In response to sTWEAK Fn14 stimulates the alternative NFκB signaling pathway and sensitizes for TNF-induced cell death (Wajant 2013). Transmembrane TWEAK triggers the same Fn14 signaling events as sTWEAK but in addition enables Fn14 to activate also the classical NFκB pathway (Wajant 2013). Hence, whether an antibody construct is an Fn14 agonist can be determined by methods known in the art, including, without limitation, methods for measuring IL8 secretion in cells expressing Fn14 (e.g. by ELISA), determining activation of the alternative NFκB pathway in cells expressing Fn14 (e.g. western blot of NIK accumulation or western blot indicating increased p100 to p52 processing) and methods for measuring TNF-induced cell death in cells expressing Fn14. Cells expressing Fn14 are known in the art and are available and include, for instance, HT-1080 cells.

The cancers to be treated according to the present invention are preferably solid cancers. A “solid cancer” is a cancer which forms one or more solid tumors. Such solid cancers forming solid tumors are generally known in the art. The term “solid cancer” encompasses both a primary tumor formed by the cancer and possible secondary tumors, which are also known as metastases. Known solid cancers to be treated according to the invention include, but are not limited to melanoma, colorectal cancer, prostate cancer, head and neck cancer, urothelial cancer, stomach cancer, pancreatic cancer, liver cancer, testis cancer, ovarian cancer, endometrial cancer, cervical cancer, brain cancer, breast cancer, gastric cancer, renal cell carcinoma, Ewing's sarcoma, non-small cell lung cancer and small cell lung cancer.

Terms such as “treatment of cancer” or “treating cancer” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). Preferably, an assessment of whether or not a therapeutic treatment works can be made based on a classification of responders and non-responders by using the response evaluation criteria in solid tumours, version 1.1 (RECIST v1.1) (Eisenhauer et al.: New response evaluation criteria in solid tumours: revised RECIST guideline (version 1.1). In: Eur. J. Cancer. 45, No. 2, January 2009, pp. 228-47). Alternatively, or additionally, an assessment of whether or not a therapeutic treatment works can be made based on known clinical indicators of cancer progression.

The treatment of cancer according to the invention can be a first-line therapy, a second-line therapy or a third-line therapy or a therapy that is beyond third-line therapy. The meaning of these terms is known in the art and in accordance with the terminology that is commonly used by the US National Cancer Institute.

Whether a cancer is a cancer expressing Fn14 can be determined by methods known in the art, including, without limitation, immunohistochemistry methods using anti-Fn14 antibodies to analyze a biopsy sample of the cancer, binding studies with TWEAK, qPCR.

In accordance with the present invention, each occurrence of the term “comprising” may optionally be substituted with the term “consisting of”.

### Methods and Techniques

Generally, unless otherwise defined herein, the methods used in the present invention (e.g. cloning methods or methods relating to antibodies) are performed in accordance with procedures known in the art, e.g. the procedures described in Sambrook et al. (“Molecular Cloning: A Laboratory Manual.”, 2nd Ed., Cold Spring Harbor Laboratory Press, Cold Spring Harbor, New York 1989), Ausubel et al. (“Current Protocols in Molecular Biology.” Greene Publishing Associates and Wiley Interscience; New York 1992), and Harlow and Lane (“Antibodies: A Laboratory Manual” Cold Spring Harbor Laboratory Press, Cold Spring Harbor, New York 1988), all of which are incorporated herein by reference.

Protein-protein binding, such as binding of antibodies to their respective target proteins, can be assessed by methods known in the art. Protein-protein binding is preferably assessed by surface plasmon resonance spectroscopy measurements.

Sequence Alignments of sequences according to the invention are performed by using the BLAST algorithm (see Altschul et al. (1990) “Basic local alignment search tool.” Journal of Molecular Biology 215. p. 403-410.; Altschul et al.: (1997) Gapped BLAST and PSI-BLAST: a new generation of protein database search programs. Nucleic Acids Res. 25:3389-3402.). Appropriate parameters for sequence alignments of short peptides by the BLAST algorithm, which are suitable for peptide antigens in accordance with the invention, are known in the art. Most software tools using the BLAST algorithm automatically adjust the parameters for sequence alignments for a short input sequence. In one embodiment, the following parameters are used: Max target sequences 10; Word size 3; BLOSUM 62 matrix; gap costs: existence 11, extension 1; conditional compositional score matrix adjustment. Thus, when used in connection with sequences, terms such as “identity” or “identical” preferably refer to the identity value obtained by using the BLAST algorithm.

### Preparation of Pharmaceutical Compositions of the Invention

Pharmaceutical compositions of the present invention are prepared in accordance with known standards for the preparation of pharmaceutical compositions. For instance, the pharmaceutical compositions are prepared in a way that they can be stored and administered appropriately. The pharmaceutical compositions of the invention may therefore comprise pharmaceutically acceptable components such as carriers, excipients and/or stabilizers. Such pharmaceutically acceptable components are not toxic in the amounts used when administering the pharmaceutical composition to a human patient. The pharmaceutical acceptable components added to the pharmaceutical compositions may depend on the chemical nature of the active ingredients present in the composition, the particular intended use of the pharmaceutical compositions and the route of administration. In general, the pharmaceutically acceptable components used in connection with the present invention are used in accordance with knowledge available in the art, e.g. from Remington's Pharmaceutical Sciences, Ed. AR Gennaro, 20th edition, 2000, Williams & Wilkins, PA, USA.

### Sequences

Preferred amino acid sequences referred to in the present application can be independently selected from the following sequences. The sequences are represented in an N-terminal to C-terminal order; and they are represented in the one-letter amino acid code.

The following non-limiting exemplary sequences were used in the experimental examples of the present application:

TABLE 1
amino acid sequences of heavy and light chain variants
of Fn14-specific antibody constructs.

| Legend: | | |
|---|---|---|
| XX | | linker sequence or internal flag tag |
| XXX | | Tenascin C (TNC) trimerization domain |
| | | (DIACGCAAAPDIKDLLSRLEELEGLVSSLREQGTG; |
| | | (SEQ ID NO: 21) |
| XXX | | const. light or const. heavy chain |
| XXX | | VH (variable heavy) or VL (variable |
| | | light domain) |
| XXX | | scFv (single-chain variable fragment) domain |
| XXX | | linker sequence of an scFv (single-chain |
| | | variable fragment) domain |
| Plasmid | | |
| No. | Plasmid name | Amino acid sequence (SEQ ID NO) |
| 1 | 18D1-HC(N297A) | EVQLVESGGGLVQPGGSLRLSCAASGFTFSNYWMSWV |
| | | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSGSSSASTKGPSVFPLAPSSKSTSGGTAALGCL |
| | | VKDYFPEPVTVSWNSGALTSGVHTFPAVLQSSGLYSLSS |
| | | WTVPSSSLGTQTYICNVNHKPSNTKVDKKVEPKSCDKT |
| | | HTCPPCPAPELLGGPSVFLFPPKPKDTLMISRTPEVTCVV |
| | | VDVSHEDPEVKFNWYVDGVEVHNAKTKPREEQYASTYR |
| | | WSVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGK |
| | | (SEQ ID NO: 1) |
| 2 | 18D1-HC(N297A)- | EVQLVESGGGLVQPGGSLRLSCAASGFTFSNYWMSWV |
| | scFv: 18D1 | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSGSSSASTKGPSVFPLAPSSKSTSGGTAALGCL |
| | | VKDYFPEPVTVSWNSGALTSGVHTFPAVLQSSGLYSLSS |
| | | WTVPSSSLGTQTYICNVNHKPSNTKVDKKVEPKSCDKT |
| | | HTCPPCPAPELLGGPSVFLFPPKPKDTLMISRTPEVTCVV |
| | | VDVSHEDPEVKFNWYVDGVEVHNAKTKPREEQYASTYR |
| | | WVSVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGKLEEVQLVES |
| | | GGGLVQPGGSLRLSCAASGFTFSNYWMSWVRQAPGKG |
| | | LEWVSGINPGGTSTYYADSVKGRFTISRDNAKNTLYLQM |
| | | NSLKSEDTAVYYCAKHLGNWGEYNYWGQGTQVTVSSR |
| | | SSTKGPKLEEGEFSEAQLQSALTQPPSVSGSPGKTVTIS |
| | | CAGTGGDVGYRNSVSWYQQLPGMAPKLLIYDVDKRAS |
| | | GITDRFSGSKSGDTASLTISGVQSEDEADYYCASQRSGI |
| | | AAVFGGGTHLTVLG |
| | | (SEQ ID NO: 2) |
| 3 | 18D1-LC | QSALTQPPSVSGSPGKTVTISCAGTGGDVGYRNSVSWY |
| | | QQLPGMAPKLLIYDVDKRASGITDRFSGSKSGDTASLTIS |
| | | GVQSEDEADYYCASQRSGIAAVFGGGTHLTVLGGSEIKR |
| | | TVAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |
| | | (SEQ ID NO: 3) |
| 4 | 18D1-LC-scFv: 18D1 | QSALTQPPSVSGSPGKTVTISCAGTGGDVGYRNSVSWY |
| | | QQLPGMAPKLLIYDVDKRASGITDRFSGSKSGDTASLTIS |
| | | GVQSEDEADYYCASQRSGIAAVFGGGTHLTVLGGSEIKR |
| | | TVAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGECLEEVQLVES |
| | | GGGLVQPGGSLRLSCAASGFTFSNYWMSWVRQAPGKG |
| | | LEWVSGINPGGTSTYYADSVKGRFTISRDNAKNTLYLQM |
| | | NSLKSEDTAVYYCAKHLGNWGEYNYWGQGTQVTVSSR |
| | | SSTKGPKLEEGEFSEAQLQSALTQPPSVSGSPGKTVTIS |
| | | CAGTGGDVGYRNSVSWYQQLPGMAPKLLIYDVDKRAS |
| | | GITDRFSGSKSGDTASLTISGVQSEDEADYYCASQRSGI |
| | | AAVFGGGTHLTVLG |
| | | (SEQ ID NO: 4) |
| 5 | scFv: 18D1-CL | EVQLVESGGGLVQPGGSLRLSCAASGFTFSNYWMSWV |
| | | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSRSSTKGPKLEEGEFSEAQLQSALTQPPSVSG |
| | | SPGKTVTISCAGTGGDVGYRNSVSWYQQLPGMAPKLLI |
| | | YDVDKRASGITDRFSGSKSGDTASLTISGVQSEDEADYY |
| | | CASQRSGIAAVFGGGTHLTVLGGSEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| | | (SEQ ID NO: 5) |
| 6 | scFv: 18D1-CH1,2,3(N297A) | EVQLVESGGGLVQPGGSLRLSCAASGFTFSNYWMSWV |
| | | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSRSSTKGPKLEEGEFSEAQLQSALTQPPSVSG |
| | | SPGKTVTISCAGTGGDVGYRNSVSWYQQLPGMAPKLLI |
| | | YDVDKRASGITDRFSGSKSGDTASLTISGVQSEDEADYY |
| | | CASQRSGIAAVFGGGTHLTVLGGSSSASTKGPSVFPLAP |
| | | SSKSTSGGTAALGCLVKDYFPEPVTVSWNSGALTSGVH |
| | | TFPAVLQSSGLYSLSSVVTVPSSSLGTQTYICNVNHKPS |
| | | NTKVDKKVEPKSCDKTHTCPPCPAPELLGGPSVFLFPPK |
| | | PKDTLMISRTPEVTCVVVDVSHEDPEVKFNWYVDGVEV |
| | | HNAKTKPREEQYASTYRVVSVLTVLHQDWLNGKEYKCK |
| | | VSNKALPAPIEKTISKAKGQPREPQVYTLPPSRDELTKNQ |
| | | VSLTCLVKGFYPSDIAVEWESNGQPENNYKTTPPVLDSD |
| | | GSFFLYSKLTVDKSRWQQGNVFSCSVMHEALHNHYTQK |
| | | SLSLSPGK |
| | | (SEQ ID NO: 6) |
| 7 | scFv: 18D1-CL-scFv: 18D1 | EVQLVESGGGLVQPGGSLRLSCAASGFTFSNYWMSWV |
| | | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSRSSTKGPKLEEGEFSEAQLQSALTQPPSVSG |
| | | SPGKTVTISCAGTGGDVGYRNSVSWYQQLPGMAPKLLI |
| | | YDVDKRASGITDRFSGSKSGDTASLTISGVQSEDEADYY |
| | | CASQRSGIAAVFGGGTHLTVLGGSEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGECLEEVQLVESGGGLVQPGGSLR |
| | | LSCAASGFTFSNYWMSWVRQAPGKGLEWVSGINPGGT |
| | | STYYADSVKGRFTISRDNAKNTLYLQMNSLKSEDTAVYY |
| | | CAKHLGNWGEYNYWGQGTQVTVSSRSSTKGPKLEEGE |
| | | FSEAQLQSALTQPPSVSGSPGKTVTISCAGTGGDVGYR |
| | | NSVSWYQQLPGMAPKLLIYDVDKRASGITDRFSGSKSG |
| | | DTASLTISGVQSEDEADYYCASQRSGIAAVFGGGTHLTV |
| | | LG |
| | | (SEQ ID NO: 7) |
| 8 | scFv: 18D1- | EVQLVESGGGLVQPGGSLRLSCAASGFTFSNYWMSWV |
| | CH_{1,2,3}(N297A)-scFv: 18D1 | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSRSSTKGPKLEEGEFSEAQLQSALTQPPSVSG |
| | | SPGKTVTISCAGTGGDVGYRNSVSWYQQLPGMAPKLLI |
| | | YDVDKRASGITDRFSGSKSGDTASLTISGVQSEDEADYY |
| | | CASQRSGIAAVFGGGTHLTVLGGSSSASTKGPSVFPLAP |
| | | SSKSTSGGTAALGCLVKDYFPEPVTVSWNSGALTSGVH |
| | | TFPAVLQSSGLYSLSSVVTVPSSSLGTQTYICNVNHKPS |
| | | NTKVDKKVEPKSCDKTHTCPPCPAPELLGGPSVFLFPPK |
| | | PKDTLMISRTPEVTCVVVDVSHEDPEVKFNWYVDGVEV |
| | | HNAKTKPREEQYASTYRVVSVLTVLHQDWLNGKEYKCK |
| | | VSNKALPAPIEKTISKAKGQPREPQVYTLPPSRDELTKNQ |
| | | VSLTCLVKGFYPSDIAVEWESNGQPENNYKTTPPVLDSD |
| | | GSFFLYSKLTVDKSRWQQGNVFSCSVMHEALHNHYTQK |
| | | SLSLSPGKLEEVQLVESGGGLVQPGGSLRLSCAASGFTF |
| | | SNYWMSWVRQAPGKGLEWVSGINPGGTSTYYADSVKG |
| | | RFTISRDNAKNTLYLQMNSLKSEDTAVYYCAKHLGNWG |
| | | EYNYWGQGTQVTVSSRSSTKGPKLEEGEFSEAQLQSA |
| | | LTQPPSVSGSPGKTVTISCAGTGGDVGYRNSVSWYQQL |
| | | PGMAPKLLIYDVDKRASGITDRFSGSKSGDTASLTISGVQ |
| | | SEDEADYYCASQRSGIAAVFGGGTHLTVLG |
| | | (SEQ ID NO: 8) |
| 9 | PDL192-HC(N297A) | EVQLVESGGGLVQPGGSLRLSCAASGFTFSSYWMSWV |
| | | RQAPGKGLEWVAEIRLKSDNYATHYAESVKGRFTISRDD |
| | | SKNSLYLQMNSLRAEDTAVYYCTGYYADAMDYWGQGT |
| | | LVTVSSGSSSASTKGPSVFPLAPSSKSTSGGTAALGCLV |
| | | KDYFPEPVTVSWNSGALTSGVHTFPAVLQSSGLYSLSSV |
| | | VTVPSSSLGTQTYICNVNHKPSNTKVDKKVEPKSCDKTH |
| | | TCPPCPAPELLGGPSVFLFPPKPKDTLMISRTPEVTCVVV |
| | | DVSHEDPEVKFNWYVDGVEVHNAKTKPREEQYASTYRV |
| | | VSVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGK |
| | | (SEQ ID NO: 9) |
| 10 | PDL192-HC(N297A)- | EVQLVESGGGLVQPGGSLRLSCAASGFTFSSYWMSWV |
| | scFv: PDL192 | RQAPGKGLEWVAEIRLKSDNYATHYAESVKGRFTISRDD |
| | | SKNSLYLQMNSLRAEDTAVYYCTGYYADAMDYWGQGT |
| | | LVTVSSGSSSASTKGPSVFPLAPSSKSTSGGTAALGCLV |
| | | KDYFPEPVTVSWNSGALTSGVHTFPAVLQSSGLYSLSSV |
| | | VTVPSSSLGTQTYICNVNHKPSNTKVDKKVEPKSCDKTH |
| | | TCPPCPAPELLGGPSVFLFPPKPKDTLMISRTPEVTCVVV |
| | | DVSHEDPEVKFNWYVDGVEVHNAKTKPREEQYASTYRV |
| | | VSVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGKEFEVQLVES |
| | | GGGLVQPGGSLRLSCAASGFTFSSYWMSWVRQAPGKG |
| | | LEWVAEIRLKSDNYATHYAESVKGRFTISRDDSKNSLYL |
| | | QMNSLRAEDTAVYYCTGYYADAMDYWGQGTLVTVSSR |
| | | SSTKGPKLEEGEFSEAQLDIQMTQSPSSLSASVGDRVTI |
| | | TCRASQSVSTSSYSYMHWYQQKPGKAPKLLIKYASNLE |
| | | SGVPSRFSGSGSGTDFTLTISSLQPEDFATYYCQHSWEI |
| | | PYTFGGGTKVEIK |
| | | (SEQ ID NO: 10) |
| 11 | PDL192-LC | DIQMTQSPSSLSASVGDRVTITCRASQSVSTSSYSYMH |
| | | WYQQKPGKAPKLLIKYASNLESGVPSRFSGSGSGTDFTL |
| | | TISSLQPEDFATYYCQHSWEIPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| | | (SEQ ID NO: 11) |
| 12 | PDL192-LC-scFv: PDL192 | DIQMTQSPSSLSASVGDRVTITCRASQSVSTSSYSYMH |
| | | WYQQKPGKAPKLLIKYASNLESGVPSRFSGSGSGTDFTL |
| | | TISSLQPEDFATYYCQHSWEIPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGECEFEVQLVESGGGLVQ |
| | | PGGSLRLSCAASGFTFSSYWMSWVRQAPGKGLEWVAE |
| | | IRLKSDNYATHYAESVKGRFTISRDDSKNSLYLQMNSLR |
| | | AEDTAVYYCTGYYADAMDYWGQGTLVTVSSRSSTKGP |
| | | KLEEGEFSEAQLDIQMTQSPSSLSASVGDRVTITCRASQ |
| | | SVSTSSYSYMHWYQQKPGKAPKLLIKYASNLESGVPSRF |
| | | SGSGSGTDFTLTISSLQPEDFATYYCQHSWEIPYTFGGG |
| | | TKVEIK |
| | | (SEQ ID NO: 12) |
| 13 | scFv: PDL 192-CL | EVQLVESGGGLVQPGGSLRLSCAASGFTFSSYWMSWV |
| | | RQAPGKGLEWVAEIRLKSDNYATHYAESVKGRFTISRDD |
| | | SKNSLYLQMNSLRAEDTAVYYCTGYYADAMDYWGQGT |
| | | LVTVSSRSSTKGPKLEEGEFSEAQLDIQMTQSPSSLSAS |
| | | VGDRVTITCRASQSVSTSSYSYMHWYQQKPGKAPKLLIK |
| | | YASNLESGVPSRFSGSGSGTDFTLTISSLQPEDFATYYC |
| | | QHSWEIPYTFGGGTKVEIKGSEIKRTVAAPSVFIFPPSDE |
| | | QLKSGTASVVCLLNNFYPREAKVQWKVDNALQSGNSQE |
| | | SVTEQDSKDSTYSLSSTLTLSKADYEKHKVYACEVTHQG |
| | | LSSPVTKSFNRGEC |
| | | (SEQ ID NO: 13) |
| 14 | scFv: PDL192- | EVQLVESGGGLVQPGGSLRLSCAASGFTFSSYWMSWV |
| | CH_{1,2,3}(N297A) | RQAPGKGLEWVAEIRLKSDNYATHYAESVKGRFTISRDD |
| | | SKNSLYLQMNSLRAEDTAVYYCTGYYADAMDYWGQGT |
| | | LVTVSSRSSTKGPKLEEGEFSEAQLDIQMTQSPSSLSAS |
| | | VGDRVTITCRASQSVSTSSYSYMHWYQQKPGKAPKLLIK |
| | | YASNLESGVPSRFSGSGSGTDFTLTISSLQPEDFATYYC |
| | | QHSWEIPYTFGGGTKVEIKGSSSASTKGPSVFPLAPSSK |
| | | STSGGTAALGCLVKDYFPEPVTVSWNSGALTSGVHTFP |
| | | AVLQSSGLYSLSSVVTVPSSSLGTQTYICNVNHKPSNTK |
| | | VDKKVEPKSCDKTHTCPPCPAPELLGGPSVFLFPPKPKD |
| | | TLMISRTPEVTCVVVDVSHEDPEVKFNWYVDGVEVHNA |
| | | KTKPREEQYASTYRVVSVLTVLHQDWLNGKEYKCKVSN |
| | | KALPAPIEKTISKAKGQPREPQVYTLPPSRDELTKNQVSL |
| | | TCLVKGFYPSDIAVEWESNGQPENNYKTTPPVLDSDGSF |
| | | FLYSKLTVDKSRWQQGNVFSCSVMHEALHNHYTQKSLS |
| | | LSPGK |
| | | (SEQ ID NO: 14) |
| 15 | 18D1-HC(N297A)-TNC | EVQLVESGGGLVQPGGSLRLSCAASGFTFSNYWMSWV |
| | | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSGSSSASTKGPSVFPLAPSSKSTSGGTAALGCL |
| | | VKDYFPEPVTVSWNSGALTSGVHTFPAVLQSSGLYSLSS |
| | | WTVPSSSLGTQTYICNVNHKPSNTKVDKKVEPKSCDKT |
| | | HTCPPCPAPELLGGPSVFLFPPKPKDTLMISRTPEVTCVV |
| | | VDVSHEDPEVKFNWYVDGVEVHNAKTKPREEQYASTYR |
| | | WSVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGKLEDIACGCA |
| | | AAPDIKDLLSRLEELEGLVSSLREQGTG |
| | | (SEQ ID NO: 15) |
| 16 | 18D1-HC(A + RGY) | EVQLVESGGGLVQPGGSLRLSCAASGFTFSNYWMSWV |
| | | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSGSSSASTKGPSVFPLAPSSKSTSGGTAALGCL |
| | | VKDYFPEPVTVSWNSGALTSGVHTFPAVLQSSGLYSLSS |
| | | WTVPSSSLGTQTYICNVNHKPSNTKVDKKVEPKSCDKT |
| | | HTCPPCPAPELLGGPSVFLFPPKPKDTLMISRTPEVTCVV |
| | | VDVSHEDPEVKFNWYVDGVEVHNAKTKPREEQYASTYR |
| | | WSVLTVLHQDWLNGKEYKCKVSNKALPAPIEKTISKAKG |
| | | QPRRPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHGALHNHYTQKYLSLSPGK |
| | | (SEQ ID NO: 16) |
| 17 | scFv: 18D1- | EVQLVESGGGLVQPGGSLRLSCAASGFTFSNYWMSWV |
| | CH_{1,2,3}(N297A)-TNC | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSRSSTKGPKLEEGEFSEAQLQSALTQPPSVSG |
| | | SPGKTVTISCAGTGGDVGYRNSVSWYQQLPGMAPKLLI |
| | | YDVDKRASGITDRFSGSKSGDTASLTISGVQSEDEADYY |
| | | CASQRSGIAAVFGGGTHLTVLGGSSSASTKGPSVFPLAP |
| | | SSKSTSGGTAALGCLVKDYFPEPVTVSWNSGALTSGVH |
| | | TFPAVLQSSGLYSLSSVVTVPSSSLGTQTYICNVNHKPS |
| | | NTKVDKKVEPKSCDKTHTCPPCPAPELLGGPSVFLFPPK |
| | | PKDTLMISRTPEVTCVVVDVSHEDPEVKFNWYVDGVEV |
| | | HNAKTKPREEQYASTYRVVSVLTVLHQDWLNGKEYKCK |
| | | VSNKALPAPIEKTISKAKGQPREPQVYTLPPSRDELTKNQ |
| | | VSLTCLVKGFYPSDIAVEWESNGQPENNYKTTPPVLDSD |
| | | GSFFLYSKLTVDKSRWQQGNVFSCSVMHEALHNHYTQK |
| | | SLSLSPGKLEDIACGCAAAPDIKDLLSRLEELEGLVSSLR |
| | | EQGTG |
| | | (SEQ ID NO: 17) |
| 18 | scFv: 18D1-TNC- | EVQLVESGGGLVQPGGSLRLSCAASGFTFSNYWMSWV |
| | Fc(N297A) | RQAPGKGLEWVSGINPGGTSTYYADSVKGRFTISRDNA |
| | | KNTLYLQMNSLKSEDTAVYYCAKHLGNWGEYNYWGQG |
| | | TQVTVSSRSSTKGPKLEEGEFSEAQLQSALTQPPSVSG |
| | | SPGKTVTISCAGTGGDVGYRNSVSWYQQLPGMAPKLLI |
| | | YDVDKRASGITDRFSGSKSGDTASLTISGVQSEDEADYY |
| | | CASQRSGIAAVFGGGTHLTVLGGSDYKDDDDKDIACGC |
| | | AAAPDIKDLLSRLEELEGLVSSLREQGTGGGSGGRGEFK |
| | | THTCPPCPAPELLGGPSVFLFPPKPKDTLMISRTPEVTCV |
| | | WDVSHEDPEVKFNWYVDGVEVHNAKTKPREEQYASTY |
| | | RWSVLTVLHQDWLNGKEYKCKVSNKALPAPIEKTISKAK |
| | | GQPREPQVYTLPPSRDELTKNQVSLTCLVKGFYPSDIAV |
| | | EWESNGQPENNYKTTPPVLDSDGSFFLYSKLTVDKSRW |
| | | QQGNVFSCSVMHEALHNHYTQKSLSLSPGK |
| | | (SEQ ID NO: 18) |

For each of the plasmids No. 1 to 17, the encoded amino acid sequence is as follows: The indicated amino acid sequence of SEQ ID NOs 1-17, respectively, is preceded by an N-terminal amino acid sequence consisting of (in an N- to C-terminal order): a leader sequence (SED ID NO: 19: MNFGFSLIFLVLVLKGVQCEVKLVPR), a first linker consisting of the amino acids QL, a flag tag (SEQ ID: 20: DYKDDDDK), and a second linker consisting of the amino acids EF.

For the plasmid No. 18, the encoded amino acid sequence is as follows: The indicated amino acid sequence of SEQ ID NO: 18 is preceded by an N-terminal amino acid sequence consisting of (in an N- to C-terminal order): a leader sequence (SEQ ID NO: 19: MNFGFSLIFLVLVLKGVQCEVKLVPR) and a linker consisting of the amino acids EF. The amino acid sequence of SEQ ID NO: 18 carries an internal flag tag (SEQ ID NO: 20: DYKDDDDK).

Note that in accordance with the invention, the above-indicated linker sequences GS, LE and EF are cloning-related amino acid linker sequences and may be independently removed from the amino acid sequences of SEQ ID NOs 1-18, or they may be independently replaced by another linker sequence comprising 1-20 amino acids.

In accordance with the invention, the scFv domain linker sequences RSSTKGPKLEEGEFSEAQL may be independently removed from the amino acid sequences of SEQ ID NOs 2, 4, 5, 6, 7, 8, 10, 12, 13, 14, 17 and 18, or they may be independently replaced by another linker sequence comprising 1-20 amino acids. In accordance with the invention, the linker sequence GGSGGRG may be removed from the amino acid sequence of SEQ ID NO: 18.

TABLE 2
Heavy and light chain composition
of Fn14-specific antibody variants.

| Antibody | Encoded by plasmid |
|---|---|
| construct | no. from Table 1 |
| 18D1-(1) | 1 + 3 |
| 18D1-(2) | 2 + 3 |
| 18D1-(3) | 1 + 4 |
| 18D1-(4) | 2 + 4 |
| 18D1-(5) | 6 + 5 |
| 18D1-(6) | 8 + 5 |
| 18D1-(7) | 6 + 7 |
| 18D1-(8) | 8 + 7 |
| PDL192-(1) | 9 + 11 |
| PDL192-(2) | 10 + 11 |
| PDL192-(3) | 9 + 12 |
| PDL192-(4) | 10 + 12 |
| PDL192-(5) | 14 + 13 |
| 18D1-IgG1(N297A)-HC: TNC | 15 + 3 |
| 18D1-IgG1(A + RGY) | 16 + 3 |
| scFv: 18D1-IgG1(N297A) | 17 + 5 |
| scFv: 18D1-TNC-Fc(N297A) | 18 |

### EXAMPLES

The present invention is further illustrated by the following non-limiting examples.

### Material and Methods

### Cell Lines and Reagents

HEK293T and HT1080 cells were from the American Type Culture Collection (ATCC) (Rockville, MD, USA) or the German Collection of Microorganisms and Cell Cultures (DSMZ) (Braunschweig, Germany). HeLa-RIPK3-FADD_{KO} cells were described elsewhere (Füllsack et al., 2019) and Kym-1 cells (Sekiguchi et al., 1985) were a kind gift of M. Sekiguchi (University of Tokyo). All cell lines were cultivated in RPMI 1640 medium (Thermo Fischer Scientific, GB) supplemented with 10% fetal bovine serum (FBS) (Thermo Fischer Scientific, GB). Expression plasmids encoding the heavy and light chains of the various recombinant proteins (Table 1) were produced by standard cloning techniques into pCR3 (Invitrogen, Germany). Antibodies used in the study were purchased from following suppliers: Sigma-Aldrich, Germany (anti-FLAG® M2, F3165; anti-β-actin, A1978; anti-NFκB p52, 05-361), Cell Signaling, GB (anti-TRAF-1, 70745), LI-COR Biosciences, Lincoln, USA (IRDye® 800CW anti-mouse IgG, 926-32210), Dako, Glostrup, Denmark (rabbit anti-mouse IgG with horseradish peroxidase (HRP) P0260, goat anti-rabbit IgG with HRP, P0448). Production and properties of the soluble TWEAK variants Flag-sTWEAK (TWEAK) and Fc-Flag-sTWEAK (Fc-TWEAK) has been described before (Roos et al., 2010) and TNF was a kind gift from Prof. Daniela Männel (University of Regensburg, Germany).

### Production and Purification of Recombinant Proteins

HEK293T cells were transiently transfected with expression plasmids (ratio 1:1) encoding the heavy and light chain variants of the antibody variant of interest (Table 2) using polyethylenimine (PEI, Polyscience Inc., Warrington, USA) essentially as described elsewhere (Kucka at al., 2021). One day after adding the medium containing PEI/DNA mixture, the latter was replaced by RPMI 1640 medium supplemented with 2% FBS containing 100 U/ml penicillin and 100 μg/ml streptomycin (Sigma-Aldrich, Germany). After additional 5-7 days supernatants were collected, cleared by centrifugation (10 min, 4600× g) and initially assayed for the presence of recombinant proteins by western blot detection (primary antibody: anti-FLAG M2; secondary antibody anti-murine IgG IRDye 800CW). The various antibody variants were purified by anti-Flag affinity chromatography as described elsewhere (Kucka et al. 2021). Concentrations and purity of purified proteins were analyzed by SDS-PAGE and silver staining of the protein gel with the Pierce Silver Stain Kit (Thermo Fischer Scientific, USA) and comparison with the protein standards of the LMW Calibration Kit for SDS Electrophoresis from Amersham (GE Healthcare). The purity and integrity of purified recombinant antibodies were furthermore analyzed using High Performance Liquid Chromatography (HPLC) (UltiMate 3000, Thermo Fischer Scientific, USA).

Analysis of p100 Processing and TRAF1 Induction

HT1080 cells were seeded in 12-well cell culture plates (GIBCO) (2×10⁵ cells/well). Next day, medium was replaced by fresh medium supplemented with the antibodies of interest. As a positive control cells were challenged with 200 ng/ml Flag-TWEAK. After 20-24 h, total cell lysates were prepared by suspending the cell pellet in Laemmli sample buffer containing 5% β-mercaptoethanol, sonification for 25 seconds and heating for 5 minutes at 95° C. Lysates were separated by SDS-PAGE and proteins were transferred to nitrocellulose for western blot analysis of p100 to p52 processing (primary antibody: anti-NFκB p52; secondary antibody: HRP-labeled goat anti-mouse IgG) and TRAF1 induction (primary antibody: rabbit anti-TRAF1; secondary antibody: HRP-labeled goat anti-rabbit IgG). Protein loads were controlled by detection of β-actin (primary antibody: anti-β-actin; secondary antibody: HRP-labeled anti-mouse IgG).

### Analysis of IL8 Induction

HT1080 cells were seeded in 96-well cell culture plates (GIBCO) (2×10⁴ cells/well) and the next day cells were challenged with reagents of interest for an additional 24 h. As a positive control, cells were stimulated with anti-Flag antibody M2 oligomerized Flag-TWEAK which mimics the activity of membrane TWEAK (Roos et al., 2010). Cell culture supernatants were collected and analyzed with respect to their IL8 content using the human IL8 ELISA Kit (BD Biosciences, Heidelberg, Germany).

### Enhancement of TNF-Induced Cell Death

To analyze the ability of the various Fn14-specific antibody variants to enhance TNF-induced toxicity, HeLa-RIPK3-FADD_{KO} cells were seeded in the 96-well cell culture plates (GIBCO) (2.5×10⁴ cells/well). The next day, the medium was replaced by fresh medium containing the Fn14-specific reagents of interest with or without 1 ng/ml of TNF (Männel, University of Regensburg, Germany). The next day, cell viability was finally analyzed by crystal violet staining. Viability values were normalized according to untreated cells (100% viability) and cells incubated with a toxic mixture of reagents (0% viability).

### Example 1: Construction of Tetra-, Hexa- and Octavalent Anti-Fn14 Variants

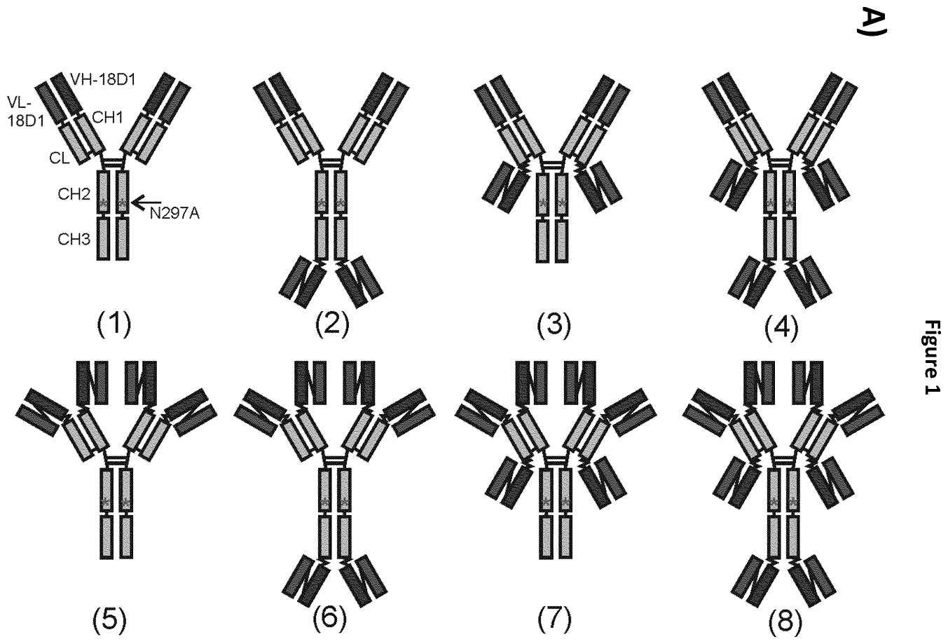

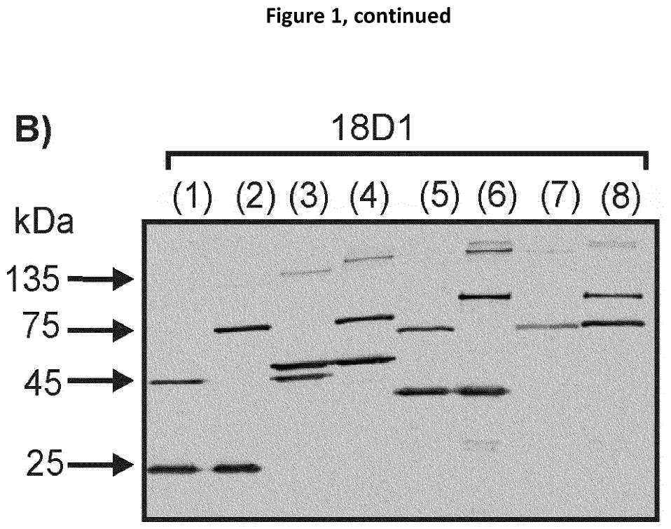

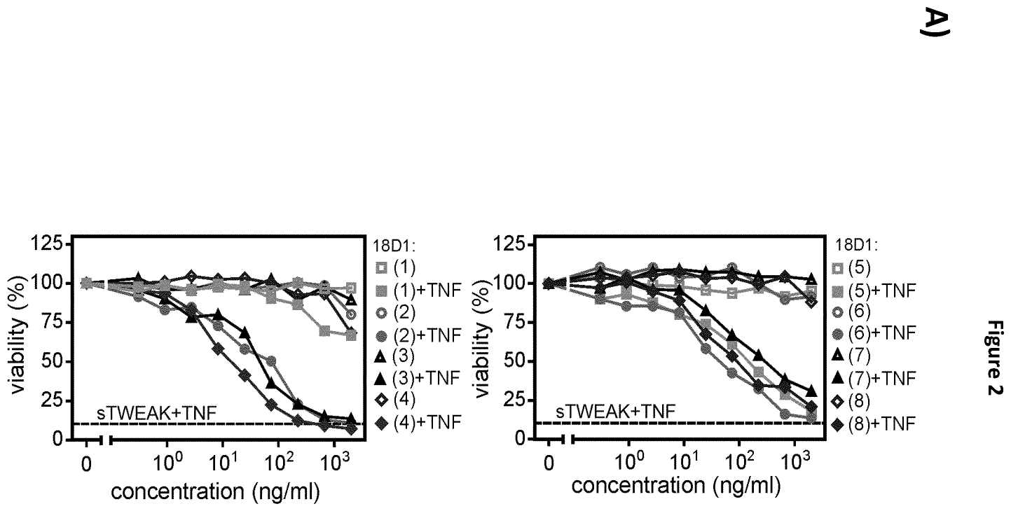

In view of the fact that anti-Fn14 antibodies can acquire memTWEAK-like activity upon crosslinking (e.g. Salzmann et al., 2013a), various multivalent variants of the anti-Fn14 antibody 18D1 (Trebing et al., 2014) were generated and analyzed the ability of these molecules to stimulate Fn14 in vitro. To obtain tetravalent 18D1 variants, a scFv domain derived of 18D1 to the C-terminus of the heavy or light chain of 18D1-IgG1 (N297A) (FIG. 1A construct 18D1-(1)) and a 18D1-IgG1 variant with a point mutation destroying/reducing FcγR binding were genetically fused, resulting in the constructs 18D1-(2) and 18D1-(3) shown in FIG. 1A. Alternatively, to have a tetravalent variant with four similarly oriented Fn14 binding sites on the same side of the antibody scaffold, the variable domains of the heavy and light chain of the parental 18D1-IgG1 antibody by scFv: 18D1 domains (FIG. 1A, construct 18D1-(5)) were replaced. Hexameric 18D1 variants were furthermore generated by fusing the scFv: 18D1 domain to the C-termini of the heavy and the light chain of 18D1-(1) (FIG. 1A, construct 18D1-(4) and by fusing this domain to the C-terminus of the heavy or light chain of construct 18D1-(5) (FIG. 1A, constructs 18D1-(6) and 18D1-(7). Finally, an octameric variant was obtained by fusing the scFv: 18D1 domain to the C-terminus of both the heavy and light chain of construct 18D1-(5) (FIG. 1A, construct 18D1-(8)). All antibody constructs were produced by transient co-transfection of HEK293 cells with expression plasmids encoding the corresponding Flag-tagged LC and HC variants. Productivity of the parental 18D1 antibody and all variants derived thereof was largely comparable (FIG. 1B).

### Example 2: Oligovalent 18D1 Variants Enhance TNF-Induced Toxicity and Trigger the Alternative NF□B Pathway but Substantially Differ in their Ability to Induce IL8

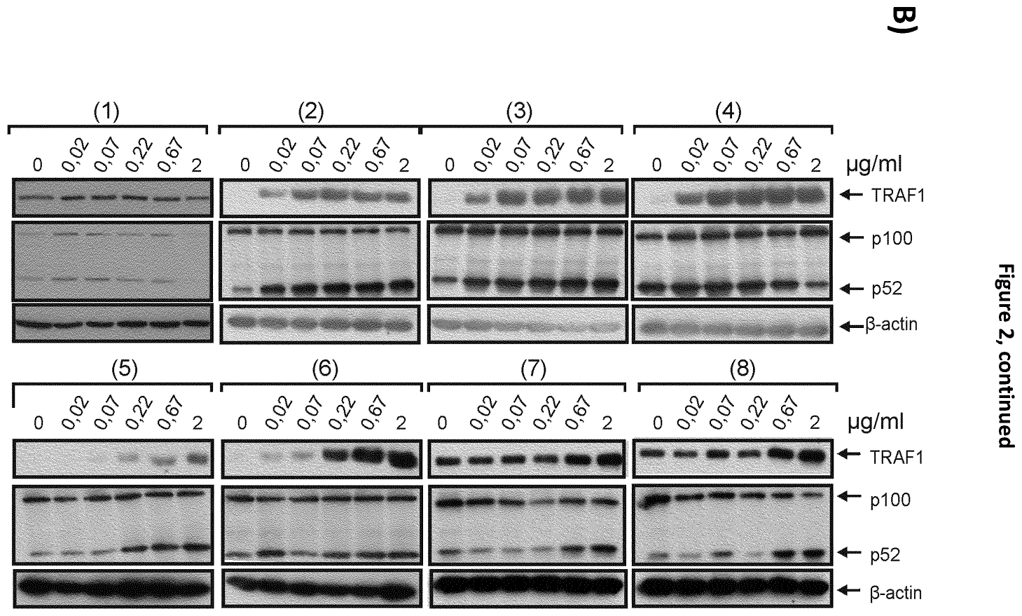

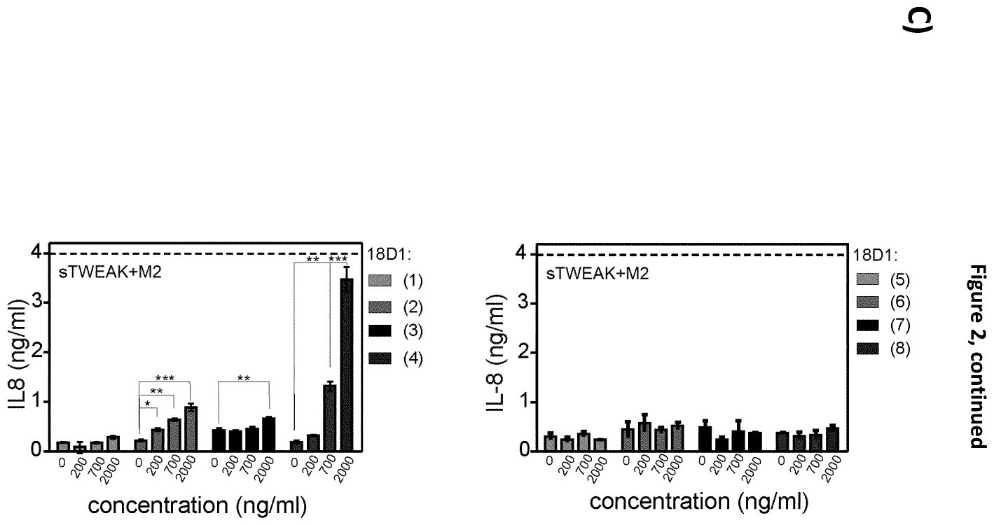

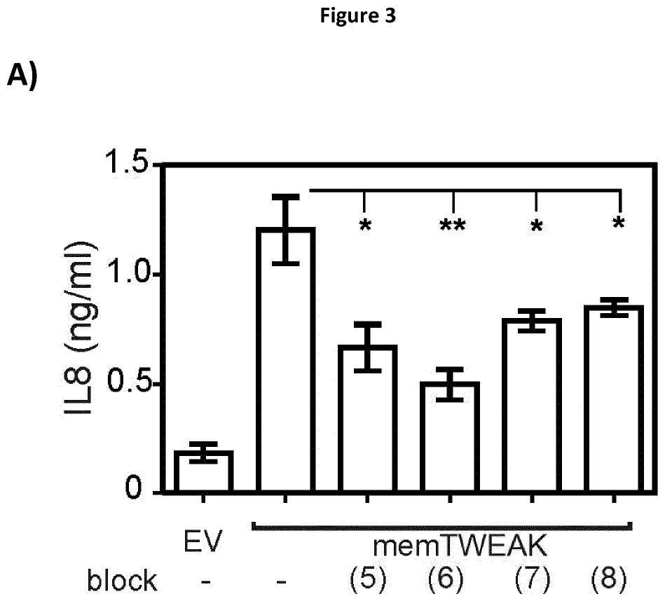

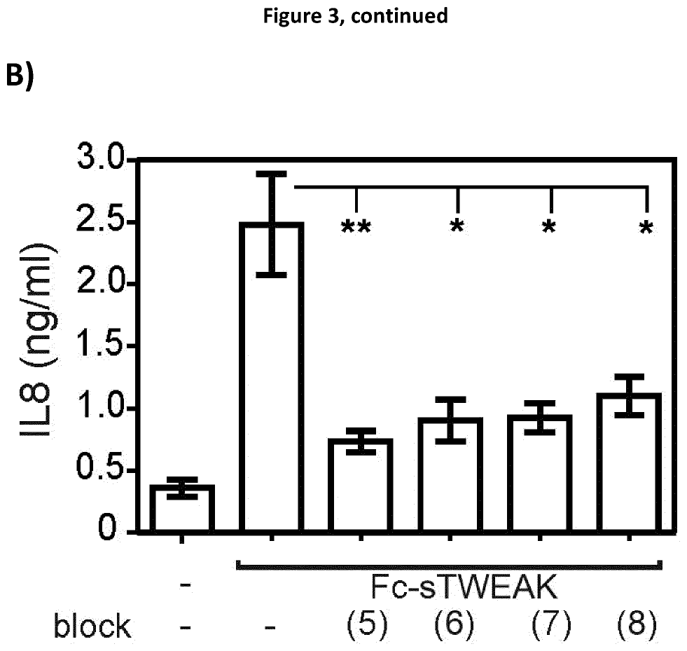

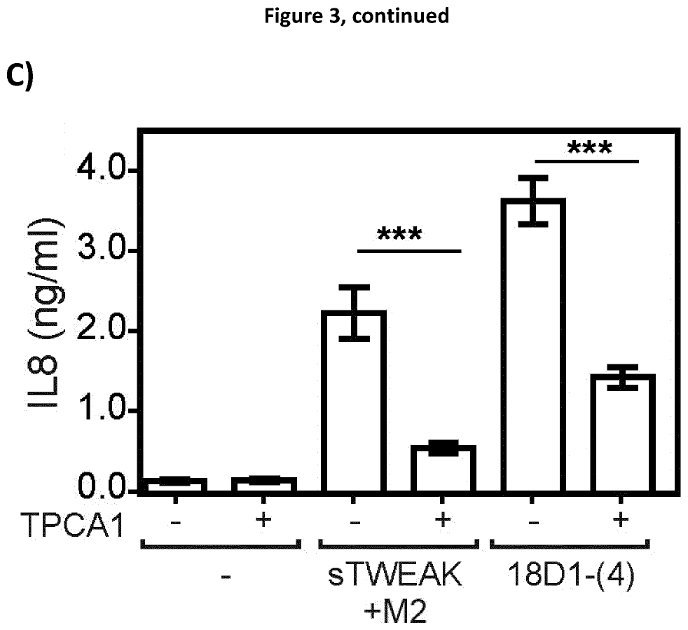

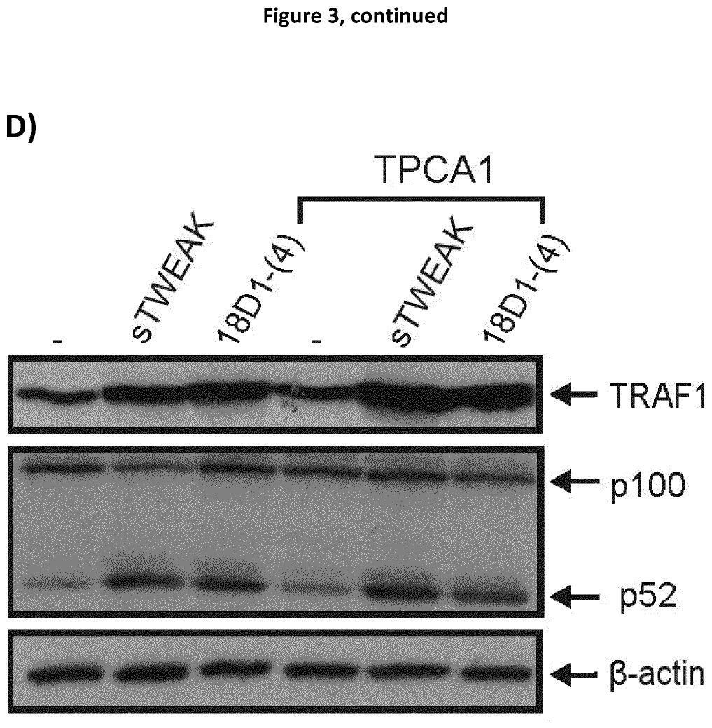

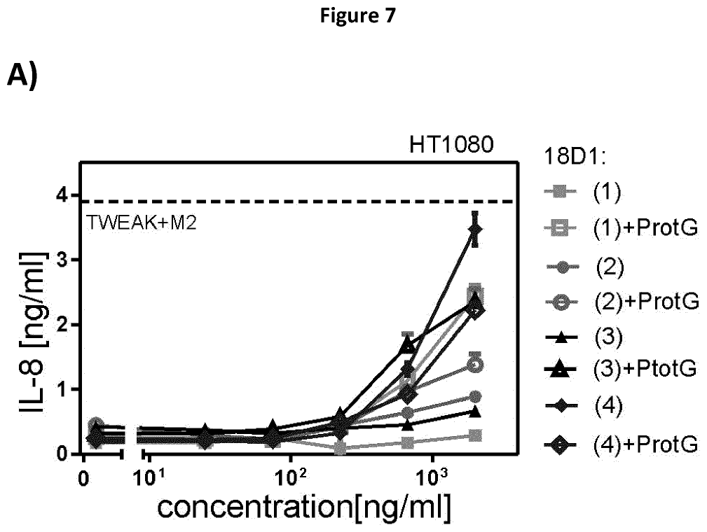

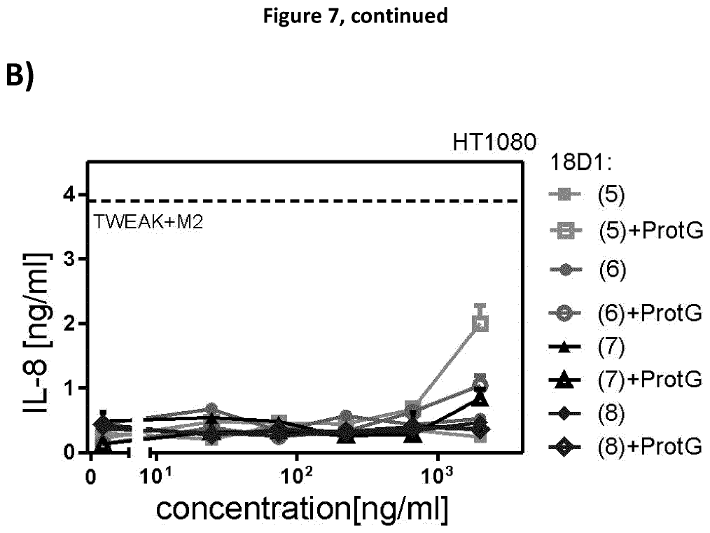

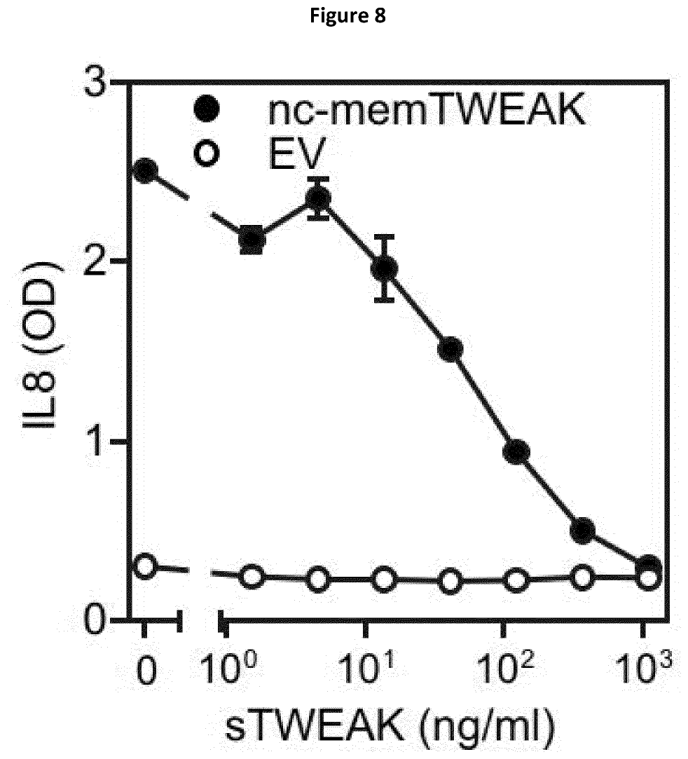

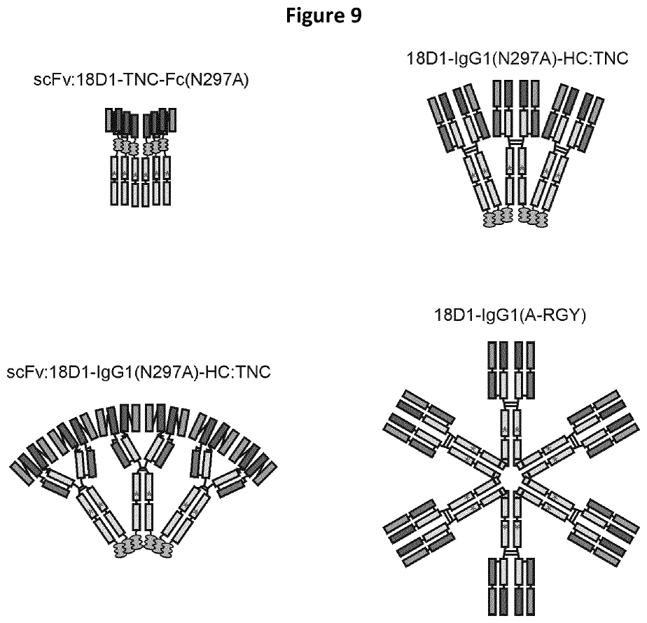

With exception of the 18D1-(1) antibody variant, all anti-Fn14 constructs enhanced TNF-induced toxicity to a comparable extent as sTWEAK with ED50-values below 100 ng/ml (FIG. 2A). All the oligovalent 18D1 constructs triggered furthermore robust p100 processing starting at concentrations of app. 20 ng/ml for 18D1-(2) to 18D1-(4) and of app. 200 ng/ml for 18D1-(5) to 18D1-(8). The conventional antibody variant (1), however, remained inactive in this respect and showed no p100 processing even at the highest concentration of 2 μg/ml (FIG. 2B). Similarly, with exception of 18D1-(1) all constructs upregulated TRAF1 expression, which is controlled by the alternative NFκB pathway (FIG. 2B). With respect to the induction of IL8, however, there was a clear difference between the constructs. IL8 is a prototypic target of the classical NFκB pathway and is accordingly not or only poorly induced by sTWEAK but efficiently by oligomerized sTWEAK and memTWEAK (Roos et al., 2010). Construct 18D1-(2) and especially construct 18D1-(4) displayed varying but significant and robust IL8 induction and reached in the case of the hexameric construct 18D1-(4) the maximum response that is induced by anti-Flag oligomerized Flag-sTWEAK (FIG. 2C). In contrast, all 18D1-derived constructs with replacement of the VH and VL domains by the scFv: 18D1 domain (18D1-(5) to 18D1-(8) remained largely inactive (FIG. 2C). These variants also showed only weak IL8 induction after protein G crosslinking although this treatment enabled the parental antibody to trigger this response (FIG. 7). Moreover, 18D1-(5) to 18D1-(8), despite their ability to trigger alternative NFκB signaling and enhancement of TNF-induced cell death (FIG. 2A,B), inhibited IL8 induction by memTWEAK expressing transfectants and hexameric Fc-sTWEAK which has memTWEAK like activity (FIG. 3A,B). In this respect, these constructs again resemble soluble TWEAK which also inhibits the memTWEAK-induced IL8 response (FIG. 8). The IKK2-specific inhibitor TPCA-1 efficiently inhibited IL8 induction by oligomerized sTWEAK and 18D1-(4) but showed no effect on p100 processing (FIG. 3C, D) confirming that Fn14-mediated IL8 induction reflected activation of the classical NFκB pathway.

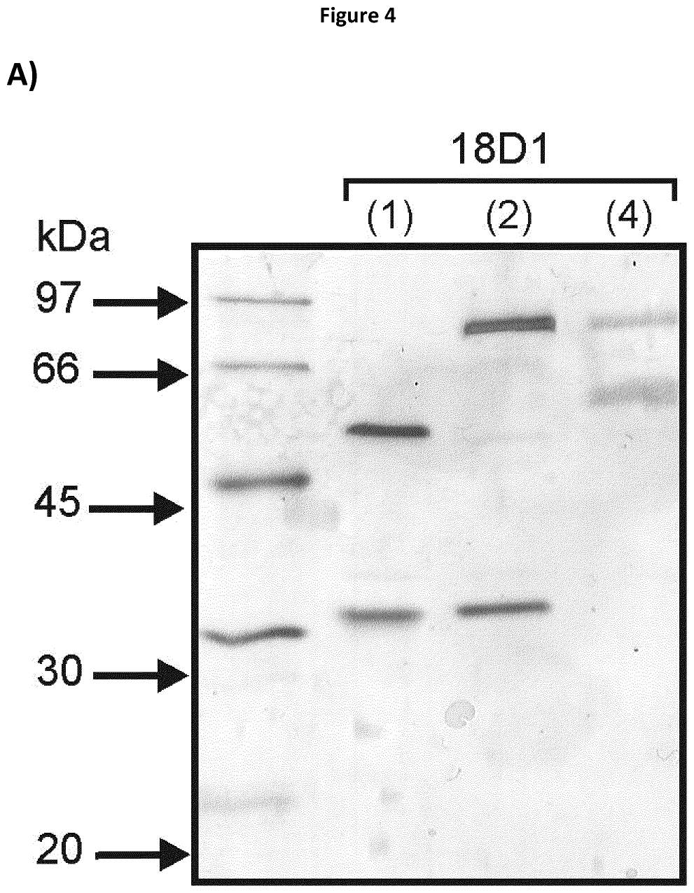

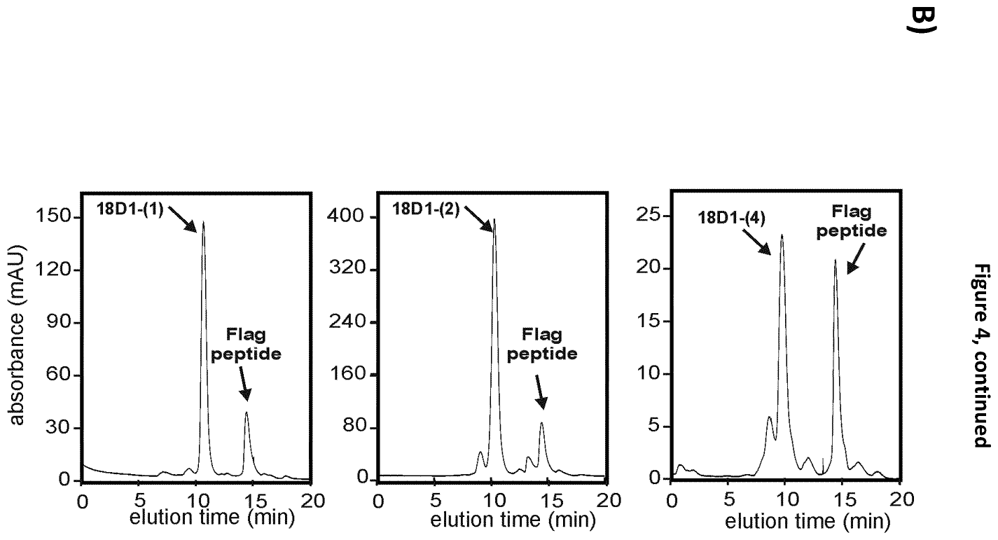

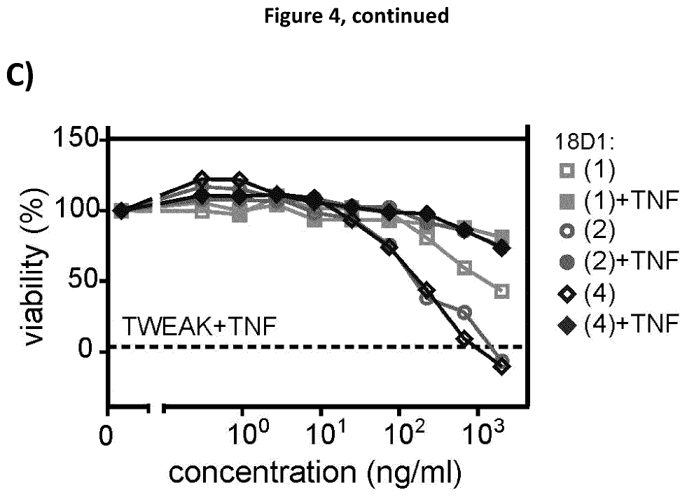

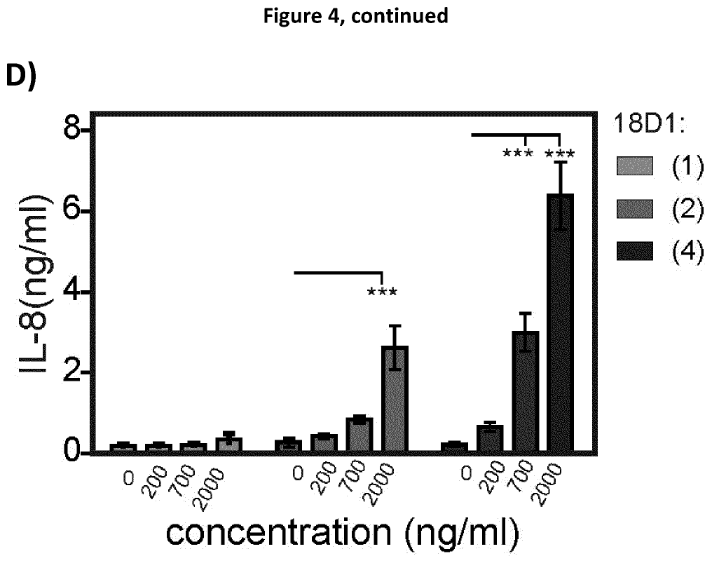

The latent agonistic activity of anti-Fn14 antibodies can be unleashed by antibody crosslinking/oligomerizing reagents such as protein G (Salzmann et al., 2013a). Therefore, to verify that the agonistic properties observed for the oligomeric 18D1 variants is not due to aggregation of the molecules and indeed mirrors intrinsic activity, the parental antibody along with the prototypic variants 18D-(2) and 18D1-(4) were purified and analyzed them by gel filtration (FIG. 4A, B). All three proteins eluted in gel filtration largely as a single molecular species with MW well corresponding to Fc domain-dimerized molecules (FIG. 4B). Functional analysis revealed furthermore that purification did not affect the functional properties of the molecules (FIG. 4C, D, E).

### Example 3: Format (2) and (4) Variants of the Anti-Fn14 mAb PDL192 Also Displays memTWEAK-Like Activity

To prove that genetic fusion of Fn14-specific scFv domains to anti-Fn14 antibodies generally favors the realization of memTWEAK mimicking agonism, construct types (2) and (4) of a second anti-Fn14 antibodies, namely PDL192, were generated and evaluated. Previous studies showed that 18D1 and PDL192 recognize different epitopes on Fn14 (Trebing et al., 2014). Moreover, the two antibodies also differ in the ability to block TWEAK binding. While 18D1 inhibits TWEAK binding, PDL192 does not compete with ligand binding (Trebing et al., 2014). Similar to the parental antibody 18D1-(1) and the 18D1-(2) variant, the parental form of PDL192 and its PDL192-(2) variant were efficiently expressed. In contrast, the expression level of the hexameric construct of type (4) was somewhat lower.

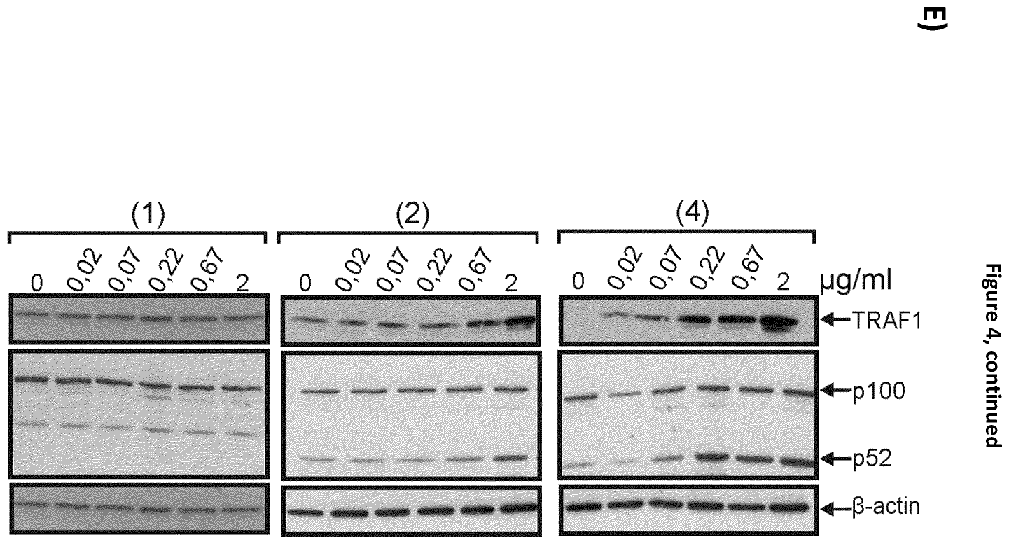

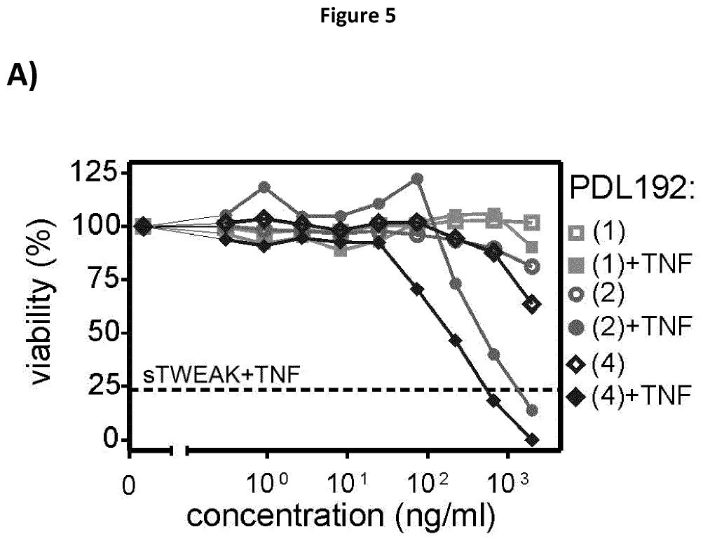

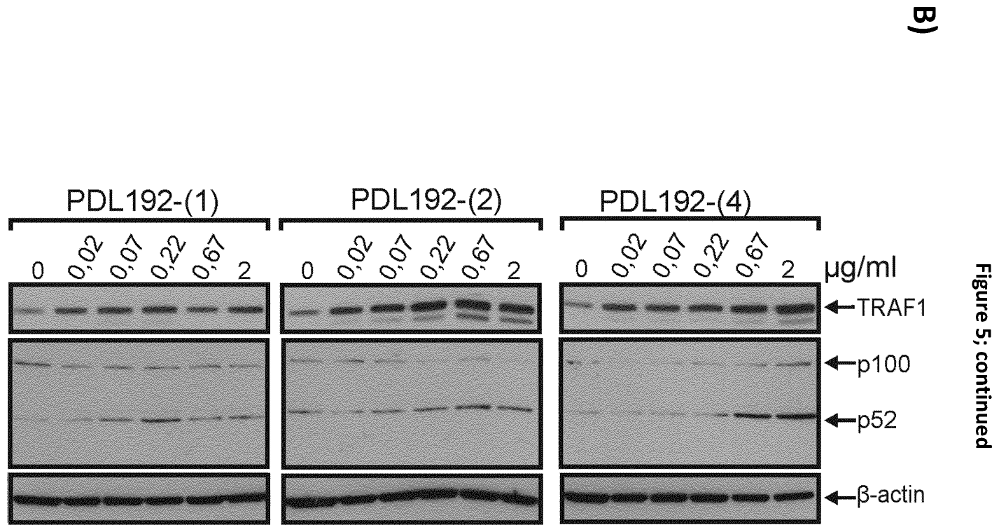

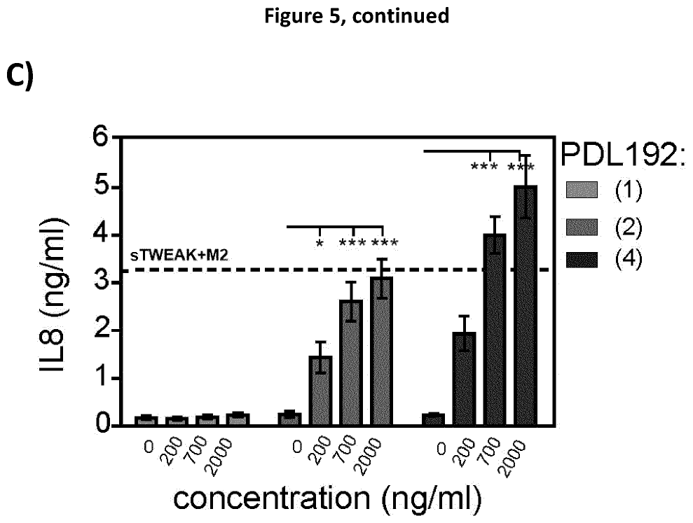

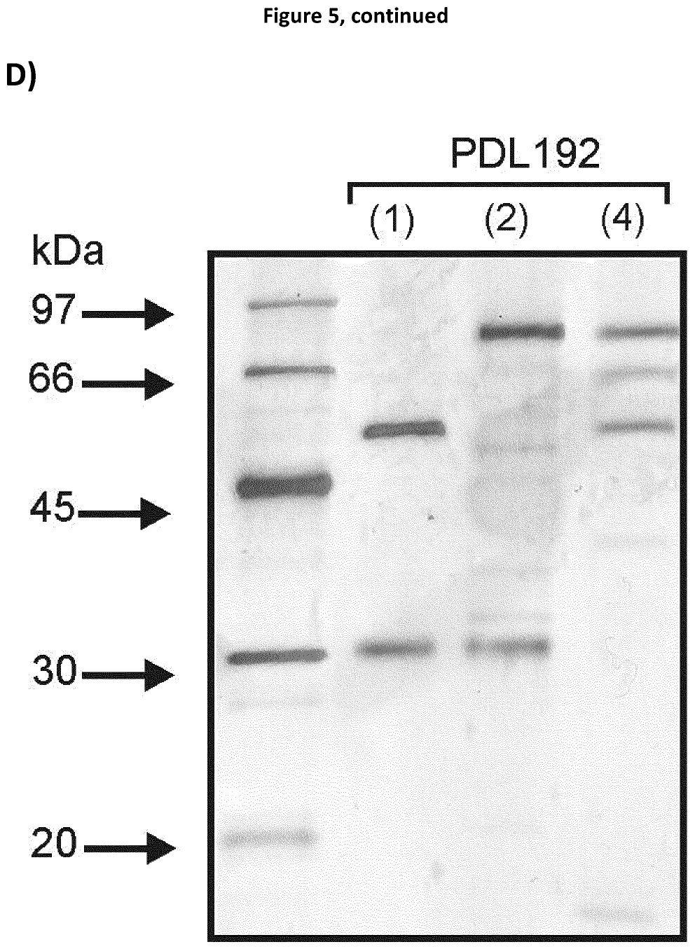

The IgG1 (N297A) version of PDL192 showed no agonism while its tetravalent and hexavalent derivatives, however, stimulated p100 processing and enhanced TNF-induced cell death (FIG. 5A, B). Both constructs also elicited strong stimulation of IL8 induction (FIG. 5C). These data suggest that type (2) and (4) antibody variants generally confer FcγR-independent memTWEAK-like agonism. To rule out again that the observed agonistic activity of the type (2) and (4) constructs of PDL192 did not result from unspecific aggregation, both constructs were purified by anti-Flag agarose affinity chromatography (FIG. 5D). All PDL192 variants proteins were efficiently purified and eluted with no evidence for aggregated high molecular weight species (FIG. 5D). Thus, the observed agonism of type (2) and type (4) anti-Fn14 variants is molecule intrinsic, too.

### Example 4: Antitumoral Activity of 18D1-(2)

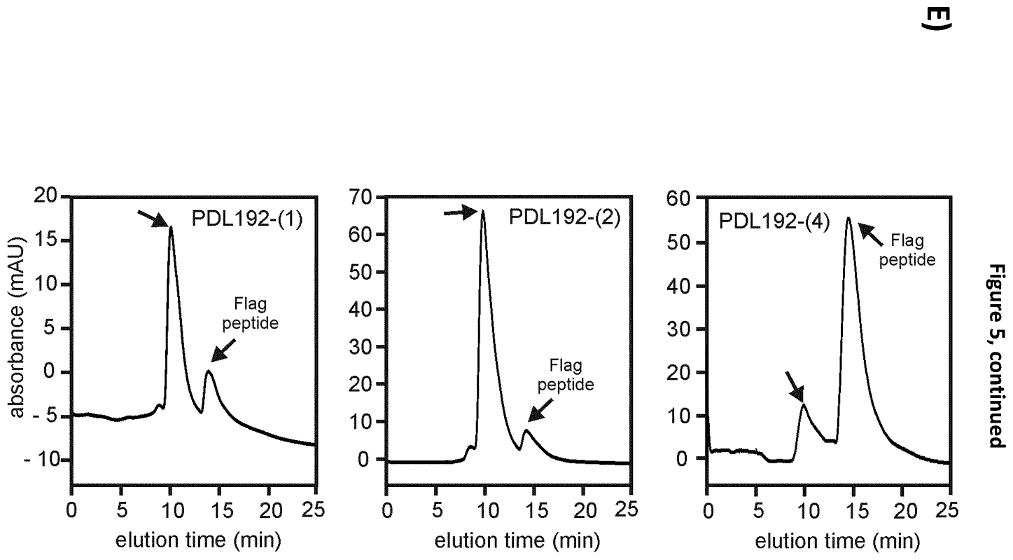

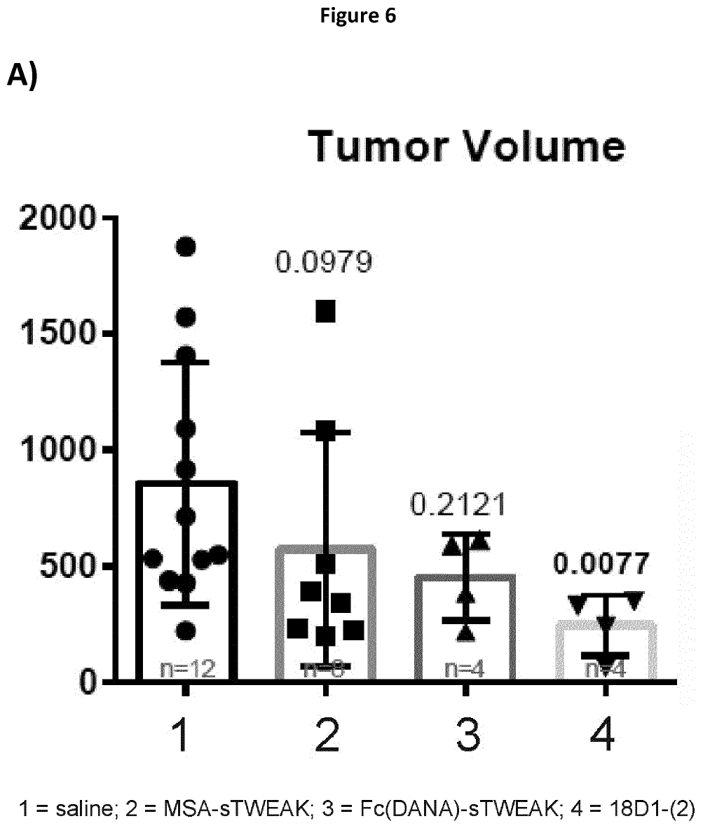

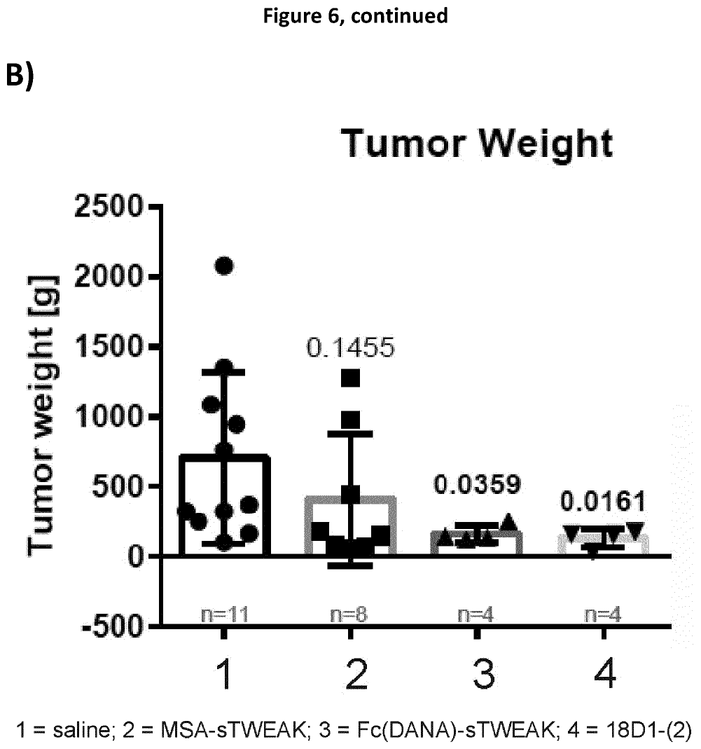

Mouse tumor organoids (MTOs), derived of tumors originated from mice with Apc^{ko/ko}, Kras^{LSL-G12D}, Tgfbr2^{ko/ko} and Trp53^{ko/ko} intestinal stem cells (Tauriello et al., 2018), were expanded in vitro and injected into the caecum wall of syngeneic C57BL/6J recipients. Two weeks after tumors were initially established, mice were treated three times per week for two weeks with 200 μg of 18D1-(2). For comparison mice were treated with MSA-sTWEAK, a fusion protein of sTWEAK with serum albumin and thus prolonged serum retention, and Fc (DANA)-sTWEAK. Tumor weight and tumor size were significantly reduced in mice treated with 18D1-(2) and there was also significantly reduced tumor weight after Fc (DANA)-sTWEAK treatment and a trend to lower tumor volume (FIG. 6). MSA-sTWEAK treated mice reached no significant antitumoral effect (FIG. 6).

### INDUSTRIAL APPLICABILITY

The pharmaceutical compositions, polypeptides, nucleic acids, cells, and products for use in the invention are industrially applicable. For example, they can be used in the manufacture of, or as, pharmaceutical products.

### REFERENCES

Meighan-Mantha R L, Hsu D K, Guo Y, Brown S A, Feng S L, Peifley K A, Alberts G F, Copeland N G, Gilbert D J, Jenkins N A, Richards C M, Winkles J A. Themitogen-inducible Fn14 gene encodes a type I transmembrane protein that modulates fibroblast adhesion and migration. J Biol Chem. 1999 Nov. 12; 274 (46): 33166-76.
Wiley S R, Cassiano L, Lofton T, Davis-Smith T, Winkles J A, Lindner V, Liu H, Daniel T O, Smith C A, Fanslow W C. A novel TNF receptor family member binds TWEAK and is implicated in angiogenesis. Immunity. 2001 November; 15 (5): 837-46.
Girgenrath M, Weng S, Kostek C A et al. TWEAK, via its receptor Fn14, is a novel regulator of mesenchymal progenitor cells and skeletal muscle regeneration. EMBO J. 2006; 25:5826-39.
Wajant H. The TWEAK-Fn14 system as a potential drug target. Br J Pharmacol. 2013 October; 170 (4): 748-64.
Chicheportiche Y, Bourdon P R, Xu H, Hsu Y M, Scott H, Hession C, Garcia I, Browning J L. TWEAK, a new secreted ligand in the tumor necrosis factor family that weakly induces apoptosis. J Biol Chem. 1997 Dec. 19; 272 (51): 32401-10.
Wu F, Guo L, Jakubowski A et al. TNF-like weak inducer of apoptosis (TWEAK) promotes beta cell neogenesis from pancreatic ductal epithelium in adult mice. PLOS One. 2013; 8: e72132.
Karaca G, Swiderska-Syn M, Xie G et al. TWEAK/Fn14 Signaling Is Required for Liver Regeneration after Partial Hepatectomy in Mice. PLOS One. 2014; 9: e83987.
Kuramitsu K, Sverdlov D Y, Liu S B et al. Failure of fibrotic liver regeneration in mice is linked to a severe fibrogenic response driven by hepatic progenitor cell activation. Am J Pathol. 2013; 183:182-94.
Mittal A, Bhatnagar S, Kumar A et al. Genetic ablation of TWEAK augments regeneration and post-injury growth of skeletal muscle in mice. Am J Pathol. 2010; 177:1732-42.
Mittal A, Bhatnagar S, Kumar A et al. The TWEAK-Fn14 system is a critical regulator of denervation-induced skeletal muscle atrophy in mice. J Cell Biol. 2010; 188:833-49.
Salzmann S, Seher A, Trebing J, Weisenberger D, Rosenthal A, Siegmund D, Wajant H. Fibroblast growth factor inducible (Fn14)-specific antibodies concomitantly display signaling pathway-specific agonistic and antagonistic activity. J Biol Chem. 2013a May 10; 288 (19): 13455-66.
Trebing J, Lang I, Chopra M, Salzmann S, Moshir M, Silence K, Riedel S S, Siegmund D, Beilhack A, Otto C, Wajant H. A novel llama antibody targeting Fn14 exhibits anti-metastatic activity in vivo. MAbs. 2014; 6:297-308.
Medler J, Nelke J, Weisenberger D, Steinfatt T, Rothaug M, Berr S, Hunig T, Beilhack A, Wajant H. TNFRSF receptor-specific antibody fusion proteins with targeting controlled FcγR-independent agonistic activity. Cell Death Dis. 2019 Mar. 4; 10 (3): 224.
Roos C, Wicovsky A, Müller N, Salzmann S, Rosenthal T, Kalthoff H, Trauzold A, Seher A, Henkler F, Kneitz C, Wajant H. Soluble and transmembrane TNF-like weak inducer of apoptosis differentially activate the classical and noncanonical NF-kappa B pathway. J Immunol. 2010 Aug. 1; 185 (3): 1593-605.
Füllsack S, Rosenthal A, Wajant H, Siegmund D. Redundant and receptor-specific activities of TRADD, RIPK1 and FADD in death receptor signaling. Cell Death Dis. 2019 Feb. 11; 10 (2): 122.
Sekiguchi M, Shiroko Y, Suzuki T, Imada M, Miyahara M, Fujii G. Characterization of a human rhabdomyosarcoma cell strain in tissue culture. Biomed Pharmacother. 1985; 39 (7): 372-80.
Kucka K, Medler J, Wajant H. Analysis of Ligand-Receptor Interactions Using Bioluminescent TNF Superfamily (TNFSF) Ligand Fusion Proteins. Methods Mol Biol. 2021; 2248:185-200.
Zhou H, Marks J W, Hittelman W N, Yagita H, Cheung L H, Rosenblum M G, Winkles J A. Development and characterization of a potent immunoconjugate targeting the Fn14 receptor on solid tumor cells. Mol Cancer Ther. 2011 July; 10 (7): 1276-88.
Zhou H, Hittelman W N, Yagita H, Cheung L H, Martin S S, Winkles J A, Rosenblum M G. Antitumor activity of a humanized, bivalent immunotoxin targeting fn14-positive solid tumors. Cancer Res. 2013 Jul. 15; 73 (14): 4439-50.
Zhou H, Mohamedali K A, Gonzalez-Angulo A M, Cao Y, Migliorini M, Cheung L H, LoBello J, Lei X, Qi Y, Hittelman W N, Winkles J A, Tran N L, Rosenblum M G. Development of human serine protease-based therapeutics targeting Fn14 and identification of Fn14 as a new target overexpressed in TNBC. Mol Cancer Ther. 2014 November; 13 (11): 2688-705.
Lerchen H G, Wittrock S, Stelte-Ludwig B, Sommer A, Berndt S, Griebenow N, Rebstock A S, Johannes S, Cancho-Grande Y, Mahlert C, Greven S, Terjung C. Antibody-Drug Conjugates with Pyrrole-Based KSP Inhibitors as the Payload Class. Angew Chem Int Ed Engl. 2018 Nov. 12; 57 (46): 15243-15247
Alvarez de Cienfuegos A, Cheung L H, Mohamedali K A, Whitsett T G, Winkles J A, Hittelman W N, Rosenblum M G. Therapeutic efficacy and safety of a human fusion construct targeting the TWEAK receptor Fn14 and containing a modified granzyme B. J Immunother Cancer. 2020 September; 8 (2): e001138
Culp P A, Choi D, Zhang Y, Yin J, Seto P, Ybarra S E, Su M, Sho M, Steinle R, Wong M H, Evangelista F, Grove J, Cardenas M, James M, Hsi E D, Chao D T, Powers D B, Ramakrishnan V, Dubridge R. Antibodies to TWEAK receptor inhibit human tumor growth through dual mechanisms. Clin Cancer Res. 2010 Jan. 15; 16 (2): 497-508.
Michaelson J S, Amatucci A, Kelly R, Su L, Garber E, Day E S, Berquist L, Cho S, Li Y, Parr M, Wille L, Schneider P, Wortham K, Burkly L C, Hsu Y M, Joseph I B. Development of an Fn14 agonistic antibody as an anti-tumor agent. MAbs. 2011 July-August; 3 (4): 362-75.
Michaelson J S, Kelly R, Yang L, Zhang X, Wortham K, Joseph I B. The anti-Fn14 antibody BIIB036 inhibits tumor growth in xenografts and patient derived primary tumor models and enhances efficacy of chemotherapeutic agents in multiple xenograft models. Cancer Biol Ther. 2012 July; 13 (9): 812-21.
Purcell J W, Kim H K, Tanlimco S G, Doan M, Fox M, Lambert P, Chao D T, Sho M, Wilson K E, Starling G C, Culp P A. Nuclear Factor κB is Required for Tumor Growth Inhibition Mediated by Enavatuzumab (PDL192), a Humanized Monoclonal Antibody to TweakR. Front Immunol. 2014 Jan. 8; 4:505.
Yin X, Luistro L, Zhong H, Smith M, Nevins T, Schostack K, Hilton H, Lin T A, Truitt T, Biondi D, Wang X, Packman K, Rosinski J, Berkofsky-Fessler W, Tang J P, Pant S, Geho D, Vega-Harring S, Demario M, Levitsky H, Simcox M. RG7212 anti-TWEAK mAb inhibits tumor growth through inhibition of tumor cell proliferation and survival signaling and by enhancing the host antitumor immune response. Clin Cancer Res. 2013 Oct. 15; 19 (20): 5686-98.
Vredevoogd D W, Kuilman T, Ligtenberg M A, Boshuizen J, Stecker K E, de Bruijn B, Krijgsman O, Huang X, Kenski J C N, Lacroix R, Mezzadra R, Gomez-Eerland R, Yildiz M, Dagidir I, Apriamashvili G, Zandhuis N, van der Noort V, Visser N L, Blank C U, Altelaar M, Schumacher T N, Peeper D S. Augmenting Immunotherapy Impact by Lowering Tumor TNF Cytotoxicity Threshold. Cell. 2019 Jul. 25; 178 (3): 585-599.e15
Litchfield K, Reading J L, Puttick C, Thakkar K, Abbosh C, Bentham R, Watkins T B K, Rosenthal R, Biswas D, Rowan A, Lim E, Al Bakir M, Turati V, Guerra-Assunção J A, Conde L, Furness A J S, Saini S K, Hadrup S R, Herrero J, Lee S H, Van Loo P, Enver T, Larkin J, Hellmann M D, Turajlic S, Quezada S A, McGranahan N, Swanton C. Meta-analysis of tumor- and T cell-intrinsic mechanisms of sensitization to checkpoint inhibition. Cell. 2021 Feb. 4; 184 (3): 596-614.e14.
Salzmann S, Lang I, Rosenthal A, Schäfer V, Weisenberger D, Carmona Arana J A, Trebing J, Siegmund D, Neumann M, Wajant H. TWEAK inhibits TRAF2-mediated CD40 signaling by destabilization of CD40 signaling complexes. J Immunol. 2013b Sep. 1; 191 (5): 2308-18.
Kucka K, Wajant H. Front Cell Dev Biol. Receptor Oligomerization and Its Relevance for Signaling by Receptors of the Tumor Necrosis Factor Receptor Superfamily. 2021 Feb. 11; 8:615141.

1. A multivalent anti-Fn14 antibody construct containing at least 4 antigen binding sites for Fn14.

2. The construct of claim 1, wherein the construct comprises an IgG molecule containing two of said antigen binding sites for Fn14.

3. The construct of claim 1, wherein the construct comprises a modified IgG molecule, which has been modified by replacing each of the two variable domains of the heavy chain by an scFv containing one of said antigen binding sites for Fn14, and by replacing each of the two variable domains of the light chain by an scFv containing one of said antigen binding sites for Fn14.

4. The construct of claim 2 or 3, wherein the construct further comprises an scFv covalently linked to the C-terminus of one of the two heavy chains of the IgG molecule, the scFv containing one of said antigen binding sites for Fn14.

5. The construct of claim 4, wherein the construct further comprises an scFv covalently linked to the C-terminus of the other of the two heavy chains of the IgG molecule, the scFv containing one of said antigen binding sites for Fn14.

6. The construct of any one of claims 2 to 5, wherein the construct further comprises an scFv covalently linked to the C-terminus of one of the two light chains of the IgG molecule, the scFv containing one of said antigen binding sites for Fn14.

7. The construct of claim 6, wherein the construct further comprises an scFv covalently linked to the C-terminus of the other of the two light chains of the IgG molecule, the scFv containing one of said antigen binding sites for Fn14.

8. The construct of any one of claims 2 to 7, wherein the IgG is IgG1, IgG2, IgG3 or IgG4.

9. The construct of any one of claims 2 to 7, wherein the IgG is IgG2 or IgG4.

10. The construct according to any one of claims 2 to 9, wherein the Fc domain of the IgG contains a mutation reducing FcγR binding.

11. The construct according to claim 10, wherein the mutation is an N297A mutation.

12. The construct according to any one of the preceding claims, wherein the construct is an Fn14 agonist.

13. The construct according to claim 12, wherein the construct is capable of activating IL8 production in cells expressing Fn14, preferably in HT-1080 cells expressing Fn14.

14. The construct according to any one of the preceding claims, wherein the construct contains at least 5 antigen binding sites for Fn14.

15. The construct according to any one of the preceding claims, wherein the construct contains at least 6 antigen binding sites for Fn14.

16. The construct according to any one of the preceding claims, wherein the construct contains 6 antigen binding sites for Fn14.

17. The construct according to any one of claims 1-2, 4-5 and 8-16, wherein the construct comprises an amino acid sequence at least 85% identical to the amino acid sequence of SEQ ID NO: 2 and an amino acid sequence at least 85% identical to the amino acid sequence of SEQ ID NO: 3, preferably an amino acid sequence at least 90% identical to the amino acid sequence of SEQ ID NO: 2 and an amino acid sequence at least 90% identical to the amino acid sequence of SEQ ID NO: 3, more preferably an amino acid sequence at least 95% identical to the amino acid sequence of SEQ ID NO: 2 and an amino acid sequence at least 95% identical to the amino acid sequence of SEQ ID NO: 3, more preferably an amino acid sequence at least 98% identical to the amino acid sequence of SEQ ID NO: 2 and an amino acid sequence at least 98% identical to the amino acid sequence of SEQ ID NO: 3, more preferably an amino acid sequence at least 99% identical to the amino acid sequence of SEQ ID NO: 2 and an amino acid sequence at least 99% identical to the amino acid sequence of SEQ ID NO: 3, and most preferably the amino acid sequence of SEQ ID NO: 2 and the amino acid sequence of SEQ ID NO: 3.

18. The construct according to any one of claims 1-2 and 6-16, wherein the construct comprises an amino acid sequence at least 85% identical to the amino acid sequence of SEQ ID NO: 1 and an amino acid sequence at least 85% identical to the amino acid sequence of SEQ ID NO: 4, preferably an amino acid sequence at least 90% identical to the amino acid sequence of SEQ ID NO: 1 and an amino acid sequence at least 90% identical to the amino acid sequence of SEQ ID NO: 4, more preferably an amino acid sequence at least 95% identical to the amino acid sequence of SEQ ID NO: 1 and an amino acid sequence at least 95% identical to the amino acid sequence of SEQ ID NO: 4, more preferably an amino acid sequence at least 98% identical to the amino acid sequence of SEQ ID NO: 1 and an amino acid sequence at least 98% identical to the amino acid sequence of SEQ ID NO: 4, more preferably an amino acid sequence at least 99% identical to the amino acid sequence of SEQ ID NO: 1 and an amino acid sequence at least 99% identical to the amino acid sequence of SEQ ID NO: 4, and most preferably the amino acid sequence of SEQ ID NO: 1 and the amino acid sequence of SEQ ID NO: 4.

19. The construct according to any one of claims 1-2 and 4-16, wherein the construct comprises an amino acid sequence at least 85% identical to the amino acid sequence of SEQ ID NO: 2 and an amino acid sequence at least 85% identical to the amino acid sequence of SEQ ID NO: 4, preferably an amino acid sequence at least 90% identical to the amino acid sequence of SEQ ID NO: 2 and an amino acid sequence at least 90% identical to the amino acid sequence of SEQ ID NO: 4, more preferably an amino acid sequence at least 95% identical to the amino acid sequence of SEQ ID NO: 2 and an amino acid sequence at least 95% identical to the amino acid sequence of SEQ ID NO: 4, more preferably an amino acid sequence at least 98% identical to the amino acid sequence of SEQ ID NO: 2 and an amino acid sequence at least 98% identical to the amino acid sequence of SEQ ID NO: 4, more preferably an amino acid sequence at least 99% identical to the amino acid sequence of SEQ ID NO: 2 and an amino acid sequence at least 99% identical to the amino acid sequence of SEQ ID NO: 4, and most preferably the amino acid sequence of SEQ ID NO: 2 and the amino acid sequence of SEQ ID NO: 4.

20. The construct according to any one of claims 1, 3 and 8-16, wherein the construct comprises an amino acid sequence at least 85% identical to the amino acid sequence of SEQ ID NO: 6 and an amino acid sequence at least 85% identical to the amino acid sequence of SEQ ID NO: 5, preferably an amino acid sequence at least 90% identical to the amino acid sequence of SEQ ID NO: 6 and an amino acid sequence at least 90% identical to the amino acid sequence of SEQ ID NO: 5, more preferably an amino acid sequence at least 95% identical to the amino acid sequence of SEQ ID NO: 6 and an amino acid sequence at least 95% identical to the amino acid sequence of SEQ ID NO: 5, more preferably an amino acid sequence at least 98% identical to the amino acid sequence of SEQ ID NO: 6 and an amino acid sequence at least 98% identical to the amino acid sequence of SEQ ID NO: 5, more preferably an amino acid sequence at least 99% identical to the amino acid sequence of SEQ ID NO: 6 and an amino acid sequence at least 99% identical to the amino acid sequence of SEQ ID NO: 5, and most preferably the amino acid sequence of SEQ ID NO: 6 and the amino acid sequence of SEQ ID NO: 5.

21. The construct according to any one of claims 1, 3-5 and 8-16, wherein the construct comprises an amino acid sequence at least 85% identical to the amino acid sequence of SEQ ID NO: 8 and an amino acid sequence at least 85% identical to the amino acid sequence of SEQ ID NO: 5, preferably an amino acid sequence at least 90% identical to the amino acid sequence of SEQ ID NO: 8 and an amino acid sequence at least 90% identical to the amino acid sequence of SEQ ID NO: 5, more preferably an amino acid sequence at least 95% identical to the amino acid sequence of SEQ ID NO: 8 and an amino acid sequence at least 95% identical to the amino acid sequence of SEQ ID NO: 5, more preferably an amino acid sequence at least 98% identical to the amino acid sequence of SEQ ID NO: 8 and an amino acid sequence at least 98% identical to the amino acid sequence of SEQ ID NO: 5, more preferably an amino acid sequence at least 99% identical to the amino acid sequence of SEQ ID NO: 8 and an amino acid sequence at least 99% identical to the amino acid sequence of SEQ ID NO: 5, and most preferably the amino acid sequence of SEQ ID NO: 8 and the amino acid sequence of SEQ ID NO: 5.

22. The construct according to any one of claims 1, 3 and 6-16, wherein the construct comprises an amino acid sequence at least 85% identical to the amino acid sequence of SEQ ID NO: 6 and an amino acid sequence at least 85% identical to the amino acid sequence of SEQ ID NO: 7, preferably an amino acid sequence at least 90% identical to the amino acid sequence of SEQ ID NO: 6 and an amino acid sequence at least 90% identical to the amino acid sequence of SEQ ID NO: 7, more preferably an amino acid sequence at least 95% identical to the amino acid sequence of SEQ ID NO: 6 and an amino acid sequence at least 95% identical to the amino acid sequence of SEQ ID NO: 7, more preferably an amino acid sequence at least 98% identical to the amino acid sequence of SEQ ID NO: 6 and an amino acid sequence at least 98% identical to the amino acid sequence of SEQ ID NO: 7, more preferably an amino acid sequence at least 99% identical to the amino acid sequence of SEQ ID NO: 6 and an amino acid sequence at least 99% identical to the amino acid sequence of SEQ ID NO: 7, and most preferably the amino acid sequence of SEQ ID NO: 6 and the amino acid sequence of SEQ ID NO: 7.

23. The construct according to any one of claims 1 and 3-16, wherein the construct comprises an amino acid sequence at least 85% identical to the amino acid sequence of SEQ ID NO: 8 and an amino acid sequence at least 85% identical to the amino acid sequence of SEQ ID NO: 7, preferably an amino acid sequence at least 90% identical to the amino acid sequence of SEQ ID NO: 8 and an amino acid sequence at least 90% identical to the amino acid sequence of SEQ ID NO: 7, more preferably an amino acid sequence at least 95% identical to the amino acid sequence of SEQ ID NO: 8 and an amino acid sequence at least 95% identical to the amino acid sequence of SEQ ID NO: 7, more preferably an amino acid sequence at least 98% identical to the amino acid sequence of SEQ ID NO: 8 and an amino acid sequence at least 98% identical to the amino acid sequence of SEQ ID NO: 7, more preferably an amino acid sequence at least 99% identical to the amino acid sequence of SEQ ID NO: 8 and an amino acid sequence at least 99% identical to the amino acid sequence of SEQ ID NO: 7, and most preferably the amino acid sequence of SEQ ID NO: 8 and the amino acid sequence of SEQ ID NO: 7.

24. The construct according to any one of claims 1-2, 4-5 and 8-16, wherein the construct comprises an amino acid sequence at least 85% identical to the amino acid sequence of SEQ ID NO: 10 and an amino acid sequence at least 85% identical to the amino acid sequence of SEQ ID NO: 11, preferably comprises an amino acid sequence at least 90% identical to the amino acid sequence of SEQ ID NO: 10 and an amino acid sequence at least 90% identical to the amino acid sequence of SEQ ID NO: 11, more preferably comprises an amino acid sequence at least 95% identical to the amino acid sequence of SEQ ID NO: 10 and an amino acid sequence at least 95% identical to the amino acid sequence of SEQ ID NO: 11, more preferably an amino acid sequence at least 98% identical to the amino acid sequence of SEQ ID NO: 10 and an amino acid sequence at least 98% identical to the amino acid sequence of SEQ ID NO: 11, more preferably an amino acid sequence at least 99% identical to the amino acid sequence of SEQ ID NO: 10 and an amino acid sequence at least 99% identical to the amino acid sequence of SEQ ID NO: 11, and most preferably the amino acid sequence of SEQ ID NO: 10 and the amino acid sequence of SEQ ID NO: 11.

25. The construct according to any one of claims 1-2 and 6-16, wherein the construct comprises an amino acid sequence at least 85% identical to the amino acid sequence of SEQ ID NO: 9 and an amino acid sequence at least 85% identical to the amino acid sequence of SEQ ID NO: 12, preferably an amino acid sequence at least 90% identical to the amino acid sequence of SEQ ID NO: 9 and an amino acid sequence at least 90% identical to the amino acid sequence of SEQ ID NO: 12, more preferably an amino acid sequence at least 95% identical to the amino acid sequence of SEQ ID NO: 9 and an amino acid sequence at least 95% identical to the amino acid sequence of SEQ ID NO: 12, more preferably an amino acid sequence at least 98% identical to the amino acid sequence of SEQ ID NO: 9 and an amino acid sequence at least 98% identical to the amino acid sequence of SEQ ID NO: 12, more preferably an amino acid sequence at least 99% identical to the amino acid sequence of SEQ ID NO: 9 and an amino acid sequence at least 99% identical to the amino acid sequence of SEQ ID NO: 12, and most preferably the amino acid sequence of SEQ ID NO: 9 and the amino acid sequence of SEQ ID NO: 12.

26. The construct according to any one of claims 1-2 and 4-16, wherein the construct comprises an amino acid sequence at least 85% identical to the amino acid sequence of SEQ ID NO: 10 and an amino acid sequence at least 85% identical to the amino acid sequence of SEQ ID NO: 12, preferably an amino acid sequence at least 90% identical to the amino acid sequence of SEQ ID NO: 10 and an amino acid sequence at least 90% identical to the amino acid sequence of SEQ ID NO: 12, more preferably an amino acid sequence at least 95% identical to the amino acid sequence of SEQ ID NO: 10 and an amino acid sequence at least 95% identical to the amino acid sequence of SEQ ID NO: 12, more preferably an amino acid sequence at least 98% identical to the amino acid sequence of SEQ ID NO: 10 and an amino acid sequence at least 98% identical to the amino acid sequence of SEQ ID NO: 12, more preferably an amino acid sequence at least 99% identical to the amino acid sequence of SEQ ID NO: 10 and an amino acid sequence at least 99% identical to the amino acid sequence of SEQ ID NO: 12, and most preferably the amino acid sequence of SEQ ID NO: 10 and the amino acid sequence of SEQ ID NO: 12.

27. The construct according to any one of claims 1, 3 and 8-16, wherein the construct comprises an amino acid sequence at least 85% identical to the amino acid sequence of SEQ ID NO: 14 and an amino acid sequence at least 85% identical to the amino acid sequence of SEQ ID NO: 13, preferably an amino acid sequence at least 90% identical to the amino acid sequence of SEQ ID NO: 14 and an amino acid sequence at least 90% identical to the amino acid sequence of SEQ ID NO: 13, more preferably an amino acid sequence at least 95% identical to the amino acid sequence of SEQ ID NO: 14 and an amino acid sequence at least 95% identical to the amino acid sequence of SEQ ID NO: 13, more preferably an amino acid sequence at least 98% identical to the amino acid sequence of SEQ ID NO: 14 and an amino acid sequence at least 98% identical to the amino acid sequence of SEQ ID NO: 13, more preferably an amino acid sequence at least 99% identical to the amino acid sequence of SEQ ID NO: 14 and an amino acid sequence at least 99% identical to the amino acid sequence of SEQ ID NO: 13, and most preferably the amino acid sequence of SEQ ID NO: 14 and the amino acid sequence of SEQ ID NO: 13.

28. The construct according to any one of the preceding claims, wherein the construct comprises a trimerization domain.

29. The construct according to claim 28, wherein the trimerization domain is a trimerization domain of tenascin C.

30. The construct according to any one of claims 28 and 29, wherein the trimerization domain comprises the amino acid sequence of SEQ ID NO: 21 or a sequence at least 70% identical thereto, preferably at least 80% identical thereto, more preferably at least 85% identical thereto, more preferably at least 90% identical thereto, more preferably at least 93% identical thereto, and more preferably at least 96% identical thereto.

31. The construct according to any one of claims 28 to 30, wherein the construct is a construct according to any one of claims 2-3, and wherein the trimerization domain is linked to the C-terminus of the constant domains of the heavy chains of the IgG.

32. The construct according to any one of claims 1, 2, 8, 10-16 and 28-31, wherein the construct comprises an amino acid sequence at least 85% identical to the amino acid sequence of SEQ ID NO: 15 and an amino acid sequence at least 85% identical to the amino acid sequence of SEQ ID NO: 3, preferably an amino acid sequence at least 90% identical to the amino acid sequence of SEQ ID NO: 15 and an amino acid sequence at least 90% identical to the amino acid sequence of SEQ ID NO: 3, more preferably an amino acid sequence at least 95% identical to the amino acid sequence of SEQ ID NO: 15 and an amino acid sequence at least 95% identical to the amino acid sequence of SEQ ID NO: 3, more preferably an amino acid sequence at least 98% identical to the amino acid sequence of SEQ ID NO: 15 and an amino acid sequence at least 98% identical to the amino acid sequence of SEQ ID NO: 3, more preferably an amino acid sequence at least 99% identical to the amino acid sequence of SEQ ID NO: 15 and an amino acid sequence at least 99% identical to the amino acid sequence of SEQ ID NO: 3, and most preferably the amino acid sequence of SEQ ID NO: 15 and the amino acid sequence of SEQ ID NO: 3.

33. The construct according to any one of claims 1, 2, 8, and 10-16, wherein the construct comprises an amino acid sequence at least 85% identical to the amino acid sequence of SEQ ID NO: 16 and an amino acid sequence at least 85% identical to the amino acid sequence of SEQ ID NO: 3, preferably an amino acid sequence at least 90% identical to the amino acid sequence of SEQ ID NO: 16 and an amino acid sequence at least 90% identical to the amino acid sequence of SEQ ID NO: 3, more preferably an amino acid sequence at least 95% identical to the amino acid sequence of SEQ ID NO: 16 and an amino acid sequence at least 95% identical to the amino acid sequence of SEQ ID NO: 3, more preferably an amino acid sequence at least 98% identical to the amino acid sequence of SEQ ID NO: 16 and an amino acid sequence at least 98% identical to the amino acid sequence of SEQ ID NO: 3, more preferably an amino acid sequence at least 99% identical to the amino acid sequence of SEQ ID NO: 16 and an amino acid sequence at least 99% identical to the amino acid sequence of SEQ ID NO: 3, and most preferably the amino acid sequence of SEQ ID NO: 16 and the amino acid sequence of SEQ ID NO: 3.

34. The construct according to any one of claims 1, 3, 8, 10-16 and 28-31, wherein the construct comprises an amino acid sequence at least 85% identical to the amino acid sequence of SEQ ID NO: 17 and an amino acid sequence at least 85% identical to the amino acid sequence of SEQ ID NO: 5, preferably an amino acid sequence at least 90% identical to the amino acid sequence of SEQ ID NO: 17 and an amino acid sequence at least 90% identical to the amino acid sequence of SEQ ID NO: 5, more preferably an amino acid sequence at least 95% identical to the amino acid sequence of SEQ ID NO: 17 and an amino acid sequence at least 95% identical to the amino acid sequence of SEQ ID NO: 5, more preferably an amino acid sequence at least 98% identical to the amino acid sequence of SEQ ID NO: 17 and an amino acid sequence at least 98% identical to the amino acid sequence of SEQ ID NO: 5, more preferably an amino acid sequence at least 99% identical to the amino acid sequence of SEQ ID NO: 17 and an amino acid sequence at least 99% identical to the amino acid sequence of SEQ ID NO: 5, and most preferably the amino acid sequence of SEQ ID NO: 17 and the amino acid sequence of SEQ ID NO: 5.

35. The construct according to any one of claims 1, 3, 8-16 and 28-30, wherein the construct comprises an amino acid sequence at least 85% identical to the amino acid sequence of SEQ ID NO: 18, preferably an amino acid sequence at least 90% identical to the amino acid sequence of SEQ ID NO: 18, more preferably an amino acid sequence at least 95% identical to the amino acid sequence of SEQ ID NO: 18, more preferably an amino acid sequence at least 98% identical to the amino acid sequence of SEQ ID NO: 18, more preferably an amino acid sequence at least 99% identical to the amino acid sequence of SEQ ID NO: 18, and most preferably the amino acid sequence of SEQ ID NO: 18.

36. The construct according to any one of the preceding claims, further comprising an N-terminal amino acid sequence comprising one or two of the following ((a) and (b)): (a) a leader sequence comprising the amino acid sequence of SEQ ID NO: 19, optionally followed by a first linker sequence, and (b) a flag tag comprising the amino acid sequence of SEQ ID NO: 20, optionally followed by a second linker sequence.

37. A pharmaceutical composition comprising the construct according to any one of the preceding claims.

38. A pharmaceutical composition according to claim 37 or a construct according to any one of claims 1 to 36, for use in the treatment of cancer.

39. The pharmaceutical composition or construct for use according to claim 38, wherein the cancer is a cancer expressing Fn14.

40. The pharmaceutical composition or construct for use according to any one of claims 38 to 39, wherein the cancer is a solid cancer.

41. A nucleic acid, or a set of nucleic acids, encoding the construct of any one of claims 1-36.

42. A recombinant cell containing a nucleic acid, or a set of nucleic acids, according to claim 41 and expressing the construct of any one of claims 1-36.

43. A method for producing a construct of any one of claims 1-36, the method comprising expressing said construct in a recombinant cell according to claim 42 from the nucleic acid or set of nucleic acids according to claim 41, wherein the method optionally further comprises purifying and formulating said construct into a pharmaceutical composition according to claim 37.
